SEQUENCE LISTING (33 sequences)

SEQ ID NO: 1 - protein, 190 aa, Vaccinia virus
GAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPKQVAGTGVQHHHHHH

SEQ ID NO: 2 - protein, 468 aa, synthetic construct
MKCSWVIFFLMAVVTGVNSEVQLEQSGAELAKPGASVKMSCKASGYTFTRYWMHWVKQRPGQGLEWIGYINPSTGYTEYNQKFKDKATLTADKSSSTVYMQLSSLTSEDSAVYYCARTTVDGYDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 468 aa, synthetic construct
MKCSWVIFFLMAVVTGVNSEVQLVQSGAEVKKPGSSVKVSCKASGYTFTRYWMHWVRQPPGKGLEWIGYINPSTGYTEYNQKFKDRATLTADKSTSTVYMELSSLRSEDTAVYYCARTTVDGYDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 238 aa, synthetic construct
MKLPVRLLVLMFWIPASSSDIVMSQSPSSLAVSAGEKVSMSCKSSQTLLNSRTRKNYLAWYQQKPGQSPKLLIYWASTRESGVPDRFTGSGSGTDFTLTISSVQAEDLAVYYCKQSYNLWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 238 aa, synthetic construct
MKLPVRLLVLMFWIPASSSDIVMTQSPLSLPVTPGEPASISCRSSQTLLNSRTRKNYLAWYQQKPGQAPRLLIYWASTRESGVPDRFSGSGSGTDFTLKISRVEAEDVAVYYCKQSYNLWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 264 aa, Vaccinia virus
TVPTMNNAKLTSTETSFNDKQKVTFTCDQGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDMPSLSNGLISGSTFSIGGVIHLSCKSGFILTGSPSSTCIDGKWNPILPTCVRSNEKFDPVDDGPDDETDLSKLSKDVVQYEQEIESLEATYHHHHHHH

SEQ ID NO: 7 - protein, 470 aa, synthetic construct
MKCSWVIFFLMAVVTGVNSEVQLLESGGGLIKPGGSLRLSCAASGFIFRDYNINWVRQAPGKGLEWLGFIRTRASGRSTEYSASVKGRFTISRDDSKNIAYLHINSLKMEDTAVYYCAKKGDSYYYMDFWGKGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 470 aa, synthetic construct
MKCSWVIFFLMAVVTGVNSEVQLLESGGGLVQPGGSLRLSCAASGFIFRDYNINWVRQAPGKGLEWLSFIRTRASGRSTEYAASVKGRFTISRDDSKNTAYLQMNSLKTEDTAVYYCAKKGDSYYYMDFWGRGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 235 aa, synthetic construct
MKLPVRLLVLMFWIPASSSDIVLTQPASVSGSPGQSITISCTGGRSDLGDSNFVSWYQQYPGKAPKLLIYQVNKRPSGVPDRFSASKSANTASLTISGLQTEDEADYFCSSYTTTSTYVFGIGTKVVVLGQPKANPTVTLFPPSSEELQANKATLVCLISDFYPGAVTVAWKADGSPVKAGVETTTPSKQSNNKYAASSYLSLTPEQWKSHRSYSCQVTHEGSTVEKTVAPTECS

SEQ ID NO: 10 - protein, 235 aa, synthetic construct
MKLPVRLLVLMFWIPASSSQSALTQPASVSGSPGQSITISCTGGRSDLGDSNFVSWYQQLPGTAPKLLIYQVNKRPSGVPDRFSASKSANTASLTISGLQAEDEADYFCSSYTTTSTYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 11 - protein, 470 aa, synthetic construct; identical to SEQ ID NO: 7

SEQ ID NO: 12 - protein, 470 aa, synthetic construct
MKCSWVIFFLMAVVTGVNSEVQLLESGGGLVQPGGSLRLSCAASGFIFRDYNINWVRQAPGKGLEWLGFIRTRASGRSTEYSASVKGRFTISRDDSKNTAYLQMNSLKTEDTAVYYCAKKGDSYYYMDFWGRGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 235 aa, synthetic construct; identical to SEQ ID NO: 9

SEQ ID NO: 14 - protein, 235 aa, synthetic construct
MKLPVRLLVLMFWIPASSSQSALTQPASVSGSPGQSITISCTGGRSDLGDSNFVSWYQQLPGTAPKLLIYQVNKRPSGVPDRFSGSKSGNTASLTISGLQAEDEADYFCSSYTTTSTYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 15 - protein, 135 aa, Vaccinia virus
VRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMNHHHHHH

SEQ ID NO: 16 - protein, 474 aa, synthetic construct
MKCSWVIFFLMAVVTGVNSEVQLEQSGSEVKKPGASVKLSCKASGYTFTSYSLGWVRQAPGQGLEWMGWINTKTGNPTYAQGFTGRFVFSLDTSVNTAYLQITSLKAEDTAVYFCAKGTFYYGWGPYYNWFDPWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 474 aa, synthetic construct
MKCSWVIFFLMAVVTGVNSQVQLVQSGAEVKKPGASVKVSCKASGYTFTSYSLGWVRQAPGQGLEWMGWINTKTGNPTYAQGFTGRFVFSLDTSVNTAYLQMNSLKTEDTAVYYCAKGTFYYGWGPYYNWFDPWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 235 aa, synthetic construct
MKLPVRLLVLMFWIPASSSDIVLTQPPSVSAAPGQKITISCSGSGSNIGRHYVSWYQQFPGTAPKILIYDNDKRPSGISDRFSGSKSGASATLDITGLQTGDEADYYCATWDTNLSGGVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 19 - protein, 235 aa, synthetic construct
MKLPVRLLVLMFWIPASSSQSVLTQPPSVSAAPGQKVTISCSGSGSNIGRHYVSWYQQLPGTAPKILIYDNDKRPSGIPDRFSGSKSGASATLGITGLQTGDEADYYCATWDTNLSGGVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 20 - protein, 211 aa, Homo sapiens
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 211 aa, synthetic construct
GPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 211 aa, synthetic construct
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 23 - protein, 211 aa, synthetic construct
GPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 24 - protein, 211 aa, Homo sapiens; identical to SEQ ID NO: 20

SEQ ID NO: 25 - protein, 211 aa, synthetic construct; identical to SEQ ID NO: 21

SEQ ID NO: 26 - protein, 211 aa, synthetic construct; identical to SEQ ID NO: 22

SEQ ID NO: 27 - protein, 211 aa, synthetic construct; identical to SEQ ID NO: 23

SEQ ID NO: 28 - protein, 6 aa, synthetic construct
HHHHHH

SEQ ID NO: 29 - protein, 142 aa, synthetic construct
MKCSWVIFFLMAVVTGVNSEVQLEQSGAELAKPGASVKMSCKASGYTFTRYWMHWVKQRPGQGLEWIGYINPSTGYTEYNQKFKDKATLTADKSSSTVYMQLSSLTSEDSAVYYCARTTVDGYDFAYWGQGTLVTVSSASTK

SEQ ID NO: 30 - protein, 142 aa, synthetic construct
MKLPVRLLVLMFWIPASSSDIVMSQSPSSLAVSAGEKVSMSCKSSQTLLNSRTRKNYLAWYQQKPGQSPKLLIYWASTRESGVPDRFTGSGSGTDFTLTISSVQAEDLAVYYCKQSYNLWTFGGGTKLEIKRTVAAPSVFIF

SEQ ID NO: 31 - protein, 146 aa, synthetic construct
MKCSWVIFFLMAVVTGVNSEVQLLESGGGLIKPGGSLRLSCAASGFIFRDYNINWVRQAPGKGLEWLGFIRTRASGRSTEYSASVKGRFTISRDDSKNIAYLHINSLKMEDTAVYYCAKKGDSYYYMDFWGKGTAVTVSSASTKGP

SEQ ID NO: 32 - protein, 146 aa, synthetic construct
MKCSWVIFFLMAVVTGVNSEVQLEQSGSEVKKPGASVKLSCKASGYTFTSYSLGWVRQAPGQGLEWMGWINTKTGNPTYAQGFTGRFVFSLDTSVNTAYLQITSLKAEDTAVYFCAKGTFYYGWGPYYNWFDPWGQGALVTVSSAS

SEQ ID NO: 33 - protein, 146 aa, synthetic construct
MKLPVRLLVLMFWIPASSSDIVLTQPPSVSAAPGQKITISCSGSGSNIGRHYVSWYQQFPGTAPKILIYDNDKRPSGISDRFSGSKSGASATLDITGLQTGDEADYYCATWDTNLSGGVFGGGTKVTVLGQPKAAPSVTLFPPSSE

(19) United States
(12) Patent Application Publication
(10) Pub. No.: US 2026/0062462 A1
(43) Pub. Date: Mar. 5, 2026

(54) Compositions and Methods for the Prevention and Treatment of Orthopoxvirus Infections

(71) Applicant: Biofactura, Inc., Frederick, MD (US)

(72) Inventors: Darryl Bacon SAMPEY, Frederick, MD (US); David James ACREE, Frederick, MD (US); Carson Alexander BRACKNA, Frederick, MD (US); Nathaniel Pineda MACAPAGAL, Frederick, MD (US); Jeffrey Neal HAUSFELD, Frederick, MD (US)

(21) Appl. No.: 19/064,808

(22) Filed: Feb. 27, 2025

Related U.S. Application Data
(63) Continuation of application No. PCT/US2023/031646, filed on Aug. 31, 2023.
(60) Provisional application No. 63/402,900, filed on Aug. 31, 2022.

(51) Int. Cl. C07K 16/08

(52) U.S. Cl. CPC C07K 16/081; C07K 2317/24; C07K 2317/92

(57) ABSTRACT

Provided herein are antibodies and functional fragments thereof for treating or preventing viral diseases, for example pox viral diseases, for example smallpox and monkey pox. Any antibody or functional fragment thereof described herein can be an engineered antibody or engineered antibody fragment. Also, provided herein are compositions and pharmaceutical compositions containing one or more antibodies or functional fragments thereof. Further provided herein are kits containing one or more antibodies or functional fragments thereof, and methods, dosing schedules, dosage amounts, and routes of administration of antibodie(s) and functional fragment(s) thereof. Finally are provided methods of making antibodies and functional fragments thereof.

### CROSS-REFERENCE TO RELATED APPLICATIONS

This application is a continuation of PCT/US2023/031646, filed Aug. 31, 2023, which claims the benefit of U.S. Provisional Application No. 63/402,900, filed Aug. 31, 2022, the entire contents of both of which are incorporated herein by reference.

### STATEMENT REGARDING FEDERALLY SPONSORED RESEARCH OR DEVELOPMENT

This invention was made with Government support under contract number 75A50119C00054 awarded by the Department of Health and Human Services; Office of the Assistant Secretary for Preparedness and Response; Biomedical Advanced Research and Development Authority. The Government has certain rights in the invention.

### SEQUENCE LISTING

The instant application contains a Sequence Listing which has been submitted electronically in ST.26 (xml) format and is hereby incorporated by reference in its entirety. Said ST.26 (xml) copy, created on 2023 Sep. 28, is named 199243-709601_SL_xml and is 38,003 bytes in size.

### SUMMARY OF THE INVENTION

Disclosed herein are compositions for the prevention and treatment of orthopoxvirus infections such as vaccinia virus, monkeypox virus and variola virus, the causative agent of smallpox disease in humans, including compositions containing an antibody that binds to an epitope found on the intracellular mature virion or mature virion forms of orthopoxviruses and/or an antibody that binds to an epitope found on the extracellular enveloped virion or enveloped virion forms of orthopoxviruses. Also disclosed are compositions such as humanized antibodies and those antibodies that have been modified to reduce immunogenicity and/or extend serum half-life, and to methods for their use in imparting passive immunity against orthopoxvirus infections to individuals at risk of orthopoxvirus infections or who exhibit vaccinia infection, including adverse events caused by certain smallpox vaccines such as progressive vaccinia and eczema vaccinatum, monkeypox infection or smallpox.

Provided herein are antibodies and fragments thereof for treating or preventing viral diseases, for example pox viral diseases, for example orthopoxviral diseases, for example smallpox and monkeypox. Also provided herein are compositions and pharmaceutical compositions containing one or more antibodies or fragments thereof. Further provided herein are kits containing one or more antibodies or fragments thereof, and methods, dosing schedules, dosage amounts, and routes of administration of antibodie(s) and fragment(s) thereof which can be contained in compositions. Finally, are provided methods of making antibodies and fragments thereof.

Further provided is a composition comprising: (A) a first antibody that binds to an epitope found on a Mature Virion (MV) form of viruses of the orthopoxvirus genus; and (B) a second antibody that binds to an epitope found on an Enveloped Virion (EV) form of viruses of the orthopoxvirus genus.

Additionally provided is a composition comprising: (A) a first antibody that binds to an epitope found on an MV form of viruses of the orthopoxvirus genus; and (B) a second antibody that binds to a first epitope found on an EV form of viruses of the orthopoxvirus genus; and (C) a third antibody that binds to a second epitope found on said EV form of viruses of the orthopoxvirus genus, wherein said first epitope is different than said second epitope.

Additionally is provided: a method of imparting passive immunity against smallpox to a subject, said method comprising administering to said subject an effective amount of a composition comprising: (A) a first antibody that binds to an epitope found on an MV form of a variola virus; and (B) a second antibody that binds to an epitope found on an EV form of a variola virus.

Further provided herein is: a method of imparting passive immunity against infection by a monkeypox virus to a subject, said method comprising administering to said subject an effective amount of a composition comprising: (A) a first antibody that binds to an epitope found on an MV form of said monkeypox virus; and (B) a second non-blood derived antibody that binds to an epitope found on an EV form of said monkeypox virus.

Provided also herein is: a method of imparting passive immunity against smallpox to a subject, said method comprising administering to said subject an effective amount of a composition comprising: (A) a first antibody that binds to an epitope found on an MV form of a variola virus; and (B) a second antibody that binds to a first epitope found on an EV form of said variola virus; and (C) a third antibody that binds to a second epitope found on said EV form of said variola virus, wherein said second epitope is different than said first epitope.

Also disclosed herein is: a method of imparting passive immunity against infection by a monkeypox virus to a subject, said method comprising administering to said subject an effective amount of a composition comprising: (A) a first antibody that binds to an epitope found on an MV form of said monkeypox virus; and (B) a second antibody that binds to a first epitope found on an EV form of said monkeypox virus; and (C) a third antibody that binds to a second epitope found on said EV form of said monkeypox virus, wherein said second epitope is different than said first epitope.

The Summary provides exemplary embodiments, and is not meant to limit in any way what is provided herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows amino acid sequences illustrating an exemplary fully humanized heavy chain h7D11 variant VH2 amino acid sequence aligned with a c7D11 murine-human chimeric sequence, noting amino acid changes made in the humanized variant VH2 in dark grey highlights. Light grey highlighting indicates framework sequences and no highlighting represents the signal peptide, CDR sequences 1, 2, and 3, and the human IgG₁ constant region.

FIG. 2 shows amino acid sequences illustrating an exemplary fully humanized light chain h7D11 variant VK3 amino acid sequence aligned with a c7D11 murine-human chimeric sequence, noting amino acid changes made in the humanized variant VK3 in dark grey highlights. Light grey highlighting indicates framework sequences and no highlighting represents the signal peptide, CDR sequences 1, 2, and 3, and the human kappa constant region.

FIG. 3 shows amino acid sequences illustrating an exemplary fully humanized heavy chain h8A variant VH3 amino acid sequence aligned with a c8A chimeric sequence, noting amino acid changes made in the humanized variant VH3 in dark grey highlights. Light grey highlighting indicates framework sequences and no highlighting represents the signal peptide, CDR sequences 1, 2, and 3, and the human IgG₁ constant region.

FIG. 4 shows amino acid sequences illustrating an exemplary fully humanized light chain h8A variant VK2 amino acid sequence aligned with a c8A chimeric sequence, noting amino acid changes made in the humanized variant VK2 in dark grey highlights. Light grey highlighting indicates framework sequences and no highlighting represents the signal peptide, CDR sequences 1, 2, and 3, and the human kappa constant region.

FIG. 5 shows amino acid sequences illustrating an exemplary fully humanized heavy chain h8A variant VH1 amino acid sequence aligned with a c8A chimeric sequence noting amino acid changes made in the humanized variant VH1 in dark grey highlights. Light grey highlighting indicates framework sequences and no highlighting represents the signal peptide, CDR sequences 1, 2, and 3, and the human IgG₁ constant region.

FIG. 6 shows amino acid sequences illustrating an exemplary fully humanized light chain h8A variant VK3 amino acid sequence aligned with a c8A chimeric sequence noting amino acid changes made in the humanized variant VK3 in dark grey highlights. Light grey highlighting indicates framework sequences and no highlighting represents the signal peptide, CDR sequences 1, 2, and 3, and the human kappa constant region.

FIG. 7 shows amino acid sequences illustrating an exemplary fully humanized heavy chain h6C variant 2 amino acid sequence aligned with a c6C chimeric sequence noting amino acid changes made in the humanized variant 2 in dark grey highlights. Light grey highlighting indicates framework sequences and no highlighting represents the signal peptide, CDR sequences 1, 2, and 3, and the human IgG₁ constant region.

FIG. 8 shows amino acid sequences illustrating an exemplary fully humanized light chain h6C variant VK2 amino acid sequence aligned with a c6C chimeric sequence noting amino acid changes made in the humanized variant VK2 in dark grey highlights. Light grey highlighting indicates framework sequences and no highlighting represents the signal peptide, CDR sequences 1, 2, and 3, and the human kappa constant region. Also highlighted in dark grey is an Asparagine residue (N) in CDR3, indicating a potential N-linked glycosylation site.

FIGS. 9A-9D show amino acid sequences illustrating several exemplary FcRn affinity enhanced variants aligned with an unmodified Fc region of h7D11 HC. No highlights indicate the CH2 domain of the Fc region. Light grey highlights indicate the CH3 domain of the Fc region. Dark grey highlights indicate the modified amino acids. Unmodified h7D11 HC Fc Region (FIG. 9A). h7D11 Fc with amino acid substitutions M280Y, S282T, and T284E (FIG. 9B). h7D11 Fc with amino acid substitutions M456L and N462S (FIG. 9C). h7D11 Fc with amino acid substitutions M280Y, S282T, T284E, M456L and N462S (FIG. 9D).

FIGS. 10A-10D show amino acid sequences illustrating several exemplary FcRn affinity enhanced variants aligned with an unmodified Fc region of h8A HC. No highlights indicate the CH2 domain of the Fc region. Light grey highlights indicate the CH3 domain of the Fc region. Dark grey highlights indicate the modified amino acids. Unmodified h8A HC Fc Region (FIG. 10A). h8A Fc with amino acid substitutions M282Y, S284T, and T286E (FIG. 10B). h8A Fc with amino acid substitutions M458L and N464S (FIG. 10C). h8A Fc with amino acid substitutions M282Y, S284T, T286E, M458L and N464S (FIG. 10D).

FIG. 11 is a dot plot illustrating group mean body weight by study day for negative control groups 1 and 2. All groups had 10 male and 10 female BALBc mice (n=20). Group 1 was no Ectromelia virus (ECTV) challenge with vehicle treatment and Group 2 was challenged with 200 PFU of ECTV with vehicle treatment.

FIG. 12 is a dot plot illustrating test material 1 group mean body weight by study day for groups 3, 4, 5, and 6. All groups had 10 male and 10 female BALBc mice (n=20). All mice were challenged with 200 PFU of ECTV on Day 0. Groups 3, 4, 5 and 6 were treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 3 was treated on Day 3 post-challenge, Group 4 was treated on Day 4 post-challenge, Group 5 was treated on Day 5 post-challenge, and Group 6 was treated on Day 6 post-challenge.

FIG. 13 is a dot plot illustrating test material 2 group mean body weight by study day for groups 7, 8, 9, and 10. All groups had 10 male and 10 female BALBc mice (n=20). All mice were challenged with 200 PFU of ECTV on Day 0. Groups 7, 8, 9 and 10 were treated with a cocktail of 5 mg/kg h7D11 (anti-L1 mAb) and 5 mg/kg h8A (anti-B5 mAb) for a 10 mg/kg total mAb cocktail dose. Group 7 was treated on Day 3 post-challenge, Group 8 was treated on Day 4 post-challenge, Group 9 was treated on Day 5 post-challenge, and Group 10 was treated on Day 6 post-challenge.

FIG. 14 is a dot plot illustrating test material 3 group mean body weight by study day for groups 11, 12, 13, and 14. All groups had 10 male and 10 female BALBc mice (n=20). All mice were challenged with 200 PFU of ECTV on Day 0. Groups 11, 12, 13 and 14 were treated with a cocktail of 5 mg/kg h7D11 (anti-L1 mAb) and 5 mg/kg h6C (anti-A33 mAb) for a 10 mg/kg total mAb cocktail dose. Group 11 was treated on Day 3 post-challenge, Group 12 was treated on Day 4 post-challenge, Group 13 was treated on Day 5 post-challenge, and Group 14 was treated on Day 6 post-challenge.

FIG. 15 is a dot plot illustrating test material 4 group mean body weight by study day for groups 15, 16, 17, and 18. All groups had 10 male and 10 female BALBc mice (n=20). All mice were challenged with 200 PFU of ECTV on Day 0. Groups 15, 16, 17 and 18 were treated with a cocktail of the original chimeric versions of the mAbs as a comparator to the humanized versions used in all other treatment groups at 5 mg/kg c7D11 (anti-L1 mAb), 5 mg/kg c8A (anti-B5 mAb) and 5 mg/kg c6C (anti-A33 mAb) for a 15 mg/kg total mAb cocktail dose. Group 15 was treated on Day 3 post-challenge, Group 16 was treated on Day 4 post-challenge, Group 17 was treated on Day 5 post-challenge, and Group 18 was treated on Day 6 post-challenge.

FIG. 16 is a dot plot illustrating test material 5 low dose group mean body weight by study day for groups 19, 20, 21, and 22. All groups had 10 male and 10 female BALBc mice (n=20). All mice were challenged with 200 PFU of ECTV on Day 0. Groups 19, 20, 21 and 22 were treated with a cocktail of 3.33 mg/kg h7D11 (anti-L1 mAb), 3.33 mg/kg h8A (anti-B5 mAb) and 3.33 mg/kg h6C (anti-A33 mAb) for a 10 mg/kg total mAb cocktail dose. Group 19 was treated on Day 3 post-challenge, Group 20 was treated on Day 4 post-challenge, Group 21 was treated on Day 5 post-challenge, and Group 22 was treated on Day 6 post-challenge.

FIG. 17 is a Kaplan-Meier Time-to-Death Plot illustrating results for set 1. All groups had 10 male and 10 female BALBc mice (n=20). All mice were challenged with 200 PFU of ECTV on Day 0. Group 2 (negative control) was treated with vehicle only on Day 3 post-challenge. Groups 3, 4, 5 and 6 were treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 3 was treated on Day 3 post-challenge, Group 4 was treated on Day 4 post-challenge, Group 5 was treated on Day 5 post-challenge, and Group 6 was treated on Day 6 post-challenge.

FIG. 18 is a Kaplan-Meier Time-to-Death Plot illustrating results for set 2. All groups had 10 male and 10 female BALBc mice (n=20). All mice were challenged with 200 PFU of ECTV on Day 0. Group 2 (negative control) was treated with vehicle only on Day 3 post-challenge. Groups 7, 8, 9 and 10 were treated with a cocktail of 5 mg/kg h7D11 (anti-L1 mAb) and 5 mg/kg h8A (anti-B5 mAb) for a 10 mg/kg total mAb cocktail dose. Group 7 was treated on Day 3 post-challenge, Group 8 was treated on Day 4 post-challenge, Group 9 was treated on Day 5 post-challenge, and Group 10 was treated on Day 6 post-challenge.

FIG. 19 is a Kaplan-Meier Time-to-Death Plot illustrating results for set 3. All groups had 10 male and 10 female BALBc mice (n=20). All mice were challenged with 200 PFU of ECTV on Day 0. Group 2 (negative control) was treated with vehicle only on Day 3 post-challenge. Groups 11, 12, 13 and 14 were treated with a cocktail of 5 mg/kg h7D11 (anti-L1 mAb) and 5 mg/kg h6C (anti-A33 mAb) for a 10 mg/kg total mAb cocktail dose. Group 11 was treated on Day 3 post-challenge, Group 12 was treated on Day 4 post-challenge, Group 13 was treated on Day 5 post-challenge, and Group 14 was treated on Day 6 post-challenge.

FIG. 20 is a Kaplan-Meier Time-to-Death Plot illustrating results for set 4. All groups had 10 male and 10 female BALBc mice (n=20). All mice were challenged with 200 PFU of ECTV on Day 0. Group 2 (negative control) was treated with vehicle only on Day 3 post-challenge. Groups 15, 16, 17 and 18 were treated with a cocktail of the original chimeric versions of the mAbs as a comparator to the humanized versions used in all other treatment groups at 5 mg/kg c7D11 (anti-L1 mAb), 5 mg/kg c8A (anti-B5 mAb) and 5 mg/kg c6C (anti-A33 mAb) for a 15 mg/kg total mAb cocktail dose. Group 15 was treated on Day 3 post-challenge, Group 16 was treated on Day 4 post-challenge, Group 17 was treated on Day 5 post-challenge, and Group 18 was treated on Day 6 post-challenge.

FIG. 21 is a Kaplan-Meier Time-to-Death Plot illustrating results for set 5. All groups had 10 male and 10 female BALBc mice (n=20). All mice were challenged with 200 PFU of ECTV on Day 0. Group 2 (negative control) was treated with vehicle only on Day 3 post-challenge. Groups 19, 20, 21 and 22 were treated with a cocktail of 3.33 mg/kg h7D11 (anti-L1 mAb), 3.33 mg/kg h8A (anti-B5 mAb) and 3.33 mg/kg h6C (anti-A33 mAb) for a 10 mg/kg total mAb cocktail dose. Group 19 was treated on Day 3 post-challenge, Group 20 was treated on Day 4 post-challenge, Group 21 was treated on Day 5 post-challenge, and Group 22 was treated on Day 6 post-challenge.

FIG. 22 is a Kaplan-Meier Time-to-Death Plot illustrating results for intervention day 3. All groups had 10 male and 10 female BALBc mice (n=20). All mice were challenged with 200 PFU of ECTV on Day 0. Group 2 (negative control) was treated with vehicle only. All Groups on this plot were treated on Day 3 post-challenge. Group 3 was treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 7 was treated with a cocktail of 5 mg/kg h7D11 (anti-L1 mAb) and 5 mg/kg h8A (anti-B5 mAb) for a 10 mg/kg total mAb cocktail dose. Group 11 was treated with a cocktail of 5 mg/kg h7D11 (anti-L1 mAb) and 5 mg/kg h6C (anti-A33 mAb) for a 10 mg/kg total mAb cocktail dose. Group 15 was treated with a cocktail of the original chimeric versions of the mAbs as a comparator to the humanized versions used in all other treatment groups at 5 mg/kg c7D11 (anti-L1 mAb), 5 mg/kg c8A (anti-B5 mAb) and 5 mg/kg c6C (anti-A33 mAb) for a 15 mg/kg total mAb cocktail dose. Group 19 was treated with a cocktail of 3.33 mg/kg h7D11 (anti-L1 mAb), 3.33 mg/kg h8A (anti-B5 mAb) and 3.33 mg/kg h6C (anti-A33 mAb) for a 10 mg/kg total mAb cocktail dose.

FIG. 23 is a Kaplan-Meier Time-to-Death Plot illustrating results for intervention day 4. All groups had 10 male and 10 female BALBc mice (n=20). All mice were challenged with 200 PFU of ECTV on Day 0. Group 2 (negative control) was treated with vehicle only on Day 3 post-challenge. All Groups other that Group 2 on this plot were treated on Day 4 post-challenge. Group 4 was treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 8 was treated with a cocktail of 5 mg/kg h7D11 (anti-L1 mAb) and 5 mg/kg h8A (anti-B5 mAb) for a 10 mg/kg total mAb cocktail dose. Group 12 was treated with a cocktail of 5 mg/kg h7D11 (anti-L1 mAb) and 5 mg/kg h6C (anti-A33 mAb) for a 10 mg/kg total mAb cocktail dose. Group 16 was treated with a cocktail of the original chimeric versions of the mAbs as a comparator to the humanized versions used in all other treatment groups at 5 mg/kg c7D11 (anti-L1 mAb), 5 mg/kg c8A (anti-B5 mAb) and 5 mg/kg c6C (anti-A33 mAb) for a 15 mg/kg total mAb cocktail dose. Group 20 was treated with a cocktail of 3.33 mg/kg h7D11 (anti-L1 mAb), 3.33 mg/kg h8A (anti-B5 mAb) and 3.33 mg/kg h6C (anti-A33 mAb) for a 10 mg/kg total mAb cocktail dose.

FIG. 24 is a Kaplan-Meier Time-to-Death Plot illustrating results for intervention day 5. All groups had 10 male and 10 female BALBc mice (n=20). All mice were challenged with 200 PFU of ECTV on Day 0. Group 2 (negative control) was treated with vehicle only on Day 3 post-challenge. All Groups other that Group 2 on this plot were treated on Day 5 post-challenge. Group 5 was treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 9 was treated with a cocktail of 5 mg/kg h7D11 (anti-L1 mAb) and 5 mg/kg h8A (anti-B5 mAb) for a 10 mg/kg total mAb cocktail dose. Group 13 was treated with a cocktail of 5 mg/kg h7D11 (anti-L1 mAb) and 5 mg/kg h6C (anti-A33 mAb) for a 10 mg/kg total mAb cocktail dose. Group 17 was treated with a cocktail of the original chimeric versions of the mAbs as a comparator to the humanized versions used in all other treatment groups at 5 mg/kg c7D11 (anti-L1 mAb), 5 mg/kg c8A (anti-B5 mAb) and 5 mg/kg c6C (anti-A33 mAb) for a 15 mg/kg total mAb cocktail dose. Group 21 was treated with a cocktail of 3.33 mg/kg h7D11 (anti-L1 mAb), 3.33 mg/kg h8A (anti-B5 mAb) and 3.33 mg/kg h6C (anti-A33 mAb) for a 10 mg/kg total mAb cocktail dose.

FIG. 25 is a Kaplan-Meier Time-to-Death Plot illustrating results for intervention day 6. All groups had 10 male and 10 female BALBc mice (n=20). All mice were challenged with 200 PFU of ECTV on Day 0. Group 2 (negative control) was treated with vehicle only on Day 3 post-challenge. All Groups other that Group 2 on this plot were treated on Day 6 post-challenge. Group 6 was treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 10 was treated with a cocktail of 5 mg/kg h7D11 (anti-L1 mAb) and 5 mg/kg h8A (anti-B5 mAb) for a 10 mg/kg total mAb cocktail dose. Group 14 was treated with a cocktail of 5 mg/kg h7D11 (anti-L1 mAb) and 5 mg/kg h6C (anti-A33 mAb) for a 10 mg/kg total mAb cocktail dose. Group 18 was treated with a cocktail of the original chimeric versions of the mAbs as a comparator to the humanized versions used in all other treatment groups at 5 mg/kg c7D11 (anti-L1 mAb), 5 mg/kg c8A (anti-B5 mAb) and 5 mg/kg c6C (anti-A33 mAb) for a 15 mg/kg total mAb cocktail dose. Group 22 was treated with a cocktail of 3.33 mg/kg h7D11 (anti-L1 mAb), 3.33 mg/kg h8A (anti-B5 mAb) and 3.33 mg/kg h6C (anti-A33 mAb) for a 10 mg/kg total mAb cocktail dose.

### SEQUENCES

(Vaccinia Virus L1 Ectodomain with 6xHis Tag)
SEQ ID NO: 1
GAAASIQTTVNTLSERISSKLEQEANASAQTKCDIEIGNFYIRQNHGCNL
TVKNMCSADADAQLDAVLSAATETYSGLTPEQKAYVPAMFTAALNIQTSV
NTVVRDFENYVKQTCNSSAVVDNKLKIQNVIIDECYGAPGSPTNLEFINT
GSSKGNCAIKALMQLTTKATTQIAPKQVAGTGVQHHHHHH
(Chimeric 7D11 [c7D11] Heavy Chain)
SEQ ID NO: 2
MKCSWVIFFLMAVVTGVNSEVQLEQSGAELAKPGASVKMSCKASGYTFTR
YWMHWVKQRPGQGLEWIGYINPSTGYTEYNQKFKDKATLTADKSSSTVYM
QLSSLTSEDSAVYYCARTTVDGYDFAYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK
(Humanized 7D11 [h7D11] Variant VH2 Heavy Chain)
SEQ ID NO: 3
MKCSWVIFFLMAVVTGVNSEVQLVQSGAEVKKPGSSVKVSCKASGYTFTR
YWMHWVRQPPGKGLEWIGYINPSTGYTEYNQKFKDRATLTADKSTSTVYM
ELSSLRSEDTAVYYCARTTVDGYDFAYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK
(Chimeric 7D11 [c7D11] Light Chain)
SEQ ID NO: 4
MKLPVRLLVLMFWIPASSSDIVMSQSPSSLAVSAGEKVSMSCKSSQTLLN
SRTRKNYLAWYQQKPGQSPKLLIYWASTRESGVPDRFTGSGSGTDFTLTI
SSVQAEDLAVYYCKQSYNLWTFGGGTKLEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC
(Humanized 7D11 [h7D11] Variant VK3 Light Chain)
SEQ ID NO: 5
MKLPVRLLVLMFWIPASSSDIVMTQSPLSLPVTPGEPASISCRSSQTLLN
SRTRKNYLAWYQQKPGQAPRLLIYWASTRESGVPDRFSGSGSGTDFTLKI
SRVEAEDVAVYYCKQSYNLWTFGGGTKVEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC
(Vaccinia Virus B5 Ectodomain with 6xHis Tag)
SEQ ID NO: 6
TVPTMNNAKLTSTETSFNDKQKVTFTCDQGYHSLDPNAVCETDKWKYENP
CKKMCTVSDYVSELYDKPLYEVNSTMTLSCNGETKYFRCEEKNGNTSWND
TVTCPNAECQPLQLEHGSCQPVKEKYSFGEYITINCDVGYEVIGASYISC
TANSWNVIPSCQQKCDMPSLSNGLISGSTFSIGGVIHLSCKSGFILTGSP
SSTCIDGKWNPILPTCVRSNEKFDPVDDGPDDETDLSKLSKDVVQYEQEI
ESLEATYHHHHHHH
(Chimeric 8A [c8A] Heavy Chain)
SEQ ID NO: 7
MKCSWVIFFLMAVVTGVNSEVQLLESGGGLIKPGGSLRLSCAASGFIFRD
YNINWVRQAPGKGLEWLGFIRTRASGRSTEYSASVKGRFTISRDDSKNIA
YLHINSLKMEDTAVYYCAKKGDSYYYMDFWGKGTAVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK
(Humanized 8A [h8A] Variant VH3 Heavy Chain)
SEQ ID NO: 8
MKCSWVIFFLMAVVTGVNSEVQLLESGGGLVQPGGSLRLSCAASGFIFRD
YNINWVRQAPGKGLEWLSFIRTRASGRSTEYAASVKGRFTISRDDSKNTA
YLQMNSLKTEDTAVYYCAKKGDSYYYMDFWGRGTAVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK
(Chimeric 8A [c8A] Light Chain)
SEQ ID NO: 9
MKLPVRLLVLMFWIPASSSDIVLTQPASVSGSPGQSITISCTGGRSDLGD
SNFVSWYQQYPGKAPKLLIYQVNKRPSGVPDRFSASKSANTASLTISGLQ
TEDEADYFCSSYTTTSTYVFGIGTKVVVLGQPKANPTVTLFPPSSEELQA
NKATLVCLISDFYPGAVTVAWKADGSPVKAGVETTTPSKQSNNKYAASSY
LSLTPEQWKSHRSYSCQVTHEGSTVEKTVAPTECS
(Humanized 8A [h8A] Variant VK2 Light Chain)
SEQ ID NO: 10
MKLPVRLLVLMFWIPASSSQSALTQPASVSGSPGQSITISCTGGRSDLGD
SNFVSWYQQLPGTAPKLLIYQVNKRPSGVPDRFSASKSANTASLTISGLQ
AEDEADYFCSSYTTTSTYVFGTGTKVTVLGQPKAAPSVTLFPPSSEELQA
NKATLVCLISDFYPGAVTVAWKADSSPVKAGVETTTPSKQSNNKYAASSY
LSLTPEQWKSHKSYSCQVTHEGSTVEKTVAPTECS
(Chimeric 8A [c8A] Heavy Chain)
SEQ ID NO: 11
MKCSWVIFFLMAVVTGVNSEVQLLESGGGLIKPGGSLRLSCAASGFIFRD
YNINWVRQAPGKGLEWLGFIRTRASGRSTEYSASVKGRFTISRDDSKNIA
YLHINSLKMEDTAVYYCAKKGDSYYYMDFWGKGTAVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK
(Humanized 8A [h8A] Variant VH1 Heavy Chain)
SEQ ID NO: 12
MKCSWVIFFLMAVVTGVNSEVQLLESGGGLVQPGGSLRLSCAASGFIFRD
YNINWVRQAPGKGLEWLGFIRTRASGRSTEYSASVKGRFTISRDDSKNTA
YLQMNSLKTEDTAVYYCAKKGDSYYYMDFWGRGTAVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK
(Chimeric 8A [c8A] Light Chain)
SEQ ID NO: 13
MKLPVRLLVLMFWIPASSSDIVLTQPASVSGSPGQSITISCTGGRSDLGD
SNFVSWYQQYPGKAPKLLIYQVNKRPSGVPDRFSASKSANTASLTISGLQ
TEDEADYFCSSYTTTSTYVFGIGTKVVVLGQPKANPTVTLFPPSSEELQA
NKATLVCLISDFYPGAVTVAWKADGSPVKAGVETTTPSKQSNNKYAASSY
LSLTPEQWKSHRSYSCQVTHEGSTVEKTVAPTECS
(Humanized 8A [h8A] Variant VK3 Light Chain)
SEQ ID NO: 14
MKLPVRLLVLMFWIPASSSQSALTQPASVSGSPGQSITISCTGGRSDLGD
SNFVSWYQQLPGTAPKLLIYQVNKRPSGVPDRFSGSKSGNTASLTISGLQ
AEDEADYFCSSYTTTSTYVFGTGTKVTVLGQPKAAPSVTLFPPSSEELQA
NKATLVCLISDFYPGAVTVAWKADSSPVKAGVETTTPSKQSNNKYAASSY
LSLTPEQWKSHKSYSCQVTHEGSTVEKTVAPTECS
(Vaccinia Virus A33 Ectodomain with 6xHis Tag)
SEQ ID NO: 15
VRLNQCMSANEAAITDAAVAVAAASSTHRKVASSTTQYDHKESCNGLYYQ
GSCYILHSDYQLFSDAKANCTAESSTLPNKSDVLITWLIDYVEDTWGSDG
NPITKTTSDYQDSDVSQEVRKYFCVKTMNHHHHHH
(Chimeric 6C [c6C] Heavy Chain)
SEQ ID NO: 16
MKCSWVIFFLMAVVTGVNSEVQLEQSGSEVKKPGASVKLSCKASGYTFTS
YSLGWVRQAPGQGLEWMGWINTKTGNPTYAQGFTGRFVFSLDTSVNTAYL
QITSLKAEDTAVYFCAKGTFYYGWGPYYNWFDPWGQGALVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHEALHNHYTQKSLSLSPGK
(Humanized 6C [h6C] Variant VH2 Heavy Chain)
SEQ ID NO: 17
MKCSWVIFFLMAVVTGVNSQVQLVQSGAEVKKPGASVKVSCKASGYTFTS
YSLGWVRQAPGQGLEWMGWINTKTGNPTYAQGFTGRFVFSLDTSVNTAYL
QMNSLKTEDTAVYYCAKGTFYYGWGPYYNWFDPWGQGALVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHEALHNHYTQKSLSLSPGK
(Chimeric 6C [c6C] Light Chain)
SEQ ID NO: 18
MKLPVRLLVLMFWIPASSSDIVLTQPPSVSAAPGQKITISCSGSGSNIGR
HYVSWYQQFPGTAPKILIYDNDKRPSGISDRFSGSKSGASATLDITGLQT
GDEADYYCATWDTNLSGGVFGGGTKVTVLGQPKAAPSVTLFPPSSEELQA
NKATLVCLISDFYPGAVTVAWKADSSPVKAGVETTTPSKQSNNKYAASSY
LSLTPEQWKSHKSYSCQVTHEGSTVEKTVAPTECS
(Humanized 6C [h6C] Variant VK2 Light Chain)
SEQ ID NO: 19
MKLPVRLLVLMFWIPASSSQSVLTQPPSVSAAPGQKVTISCSGSGSNIGR
HYVSWYQQLPGTAPKILIYDNDKRPSGIPDRFSGSKSGASATLGITGLQT
GDEADYYCATWDTNLSGGVFGGGTKLTVLGQPKAAPSVTLFPPSSEELQA
NKATLVCLISDFYPGAVTVAWKADSSPVKAGVETTTPSKQSNNKYAASSY
LSLTPEQWKSHKSYSCQVTHEGSTVEKTVAPTECS
(h7D11 Fc Unmodified)
SEQ ID NO: 20
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK
(h7D11 Fc YTE Swap)
SEQ ID NO: 21
GPSVFLFPPKPKDTLYITREPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK
(h7D11 Fc LS Swap)
SEQ ID NO: 22
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVLHEALHSHY
TQKSLSLSPGK
(h7D11 Fc YTELS Swap)
SEQ ID NO: 23
GPSVFLFPPKPKDTLYITREPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVLHEALHSHY
TQKSLSLSPGK
(h8A Fc Unmodified)
SEQ ID NO: 24
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK
(h8A Fc YTE Swap)
SEQ ID NO: 25
GPSVFLFPPKPKDTLYITREPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK
(h8A Fc LS Swap)
SEQ ID NO: 26
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVLHEALHSHY
TQKSLSLSPGK
(h8A Fc YTELS Swap)
SEQ ID NO: 27
GPSVFLFPPKPKDTLYITREPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVLHEALHSHY
TQKSLSLSPGK

### Definitions

Unless otherwise defined, all terms used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein. In case of conflict, the present document, including definitions, will control.

In describing the disclosure, it is to be understood that a number of techniques and steps are disclosed. Each of these has individual benefit and each can also be used in conjunction with one or more, or in some cases all, of the other disclosed techniques. Methods and materials are described below, although methods and materials similar or equivalent to those described herein can be used in practice or testing of the present disclosure. Accordingly, for the sake of clarity, this description will refrain from repeating every possible combination of the individual steps. Nevertheless, the specification and claims should be read with the understanding that such combinations are entirely within the scope of the disclosure and the claims.

The terminology used herein is for the purpose of describing particular embodiments only and is not intended to be limiting of the disclosure. As used herein, the term “and/or” can include any and all combinations of one or more of the associated listed items. As used herein, the singular forms “a,” “an,” and “the” can include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. The terms “comprise(s),” “include(s),” “having,” “has,” “can,” “contain(s),” and variants thereof, as used herein, can be open-ended transitional phrases, terms, or words that do not preclude the possibility of additional acts or structures. All definitions contained herein are understood to include the plural form as well unless the context clearly dictates otherwise.

For the recitation of numeric ranges herein, each intervening number there between with the same degree of precision can be explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” can mean within an acceptable error range for the particular value as determined by one of ordinary skill in the art, which can depend in part on how the value is measured or determined, e.g., the limitations of the measurement system. For example, “about” means plus or minus 10%, per the practice in the art. Alternatively, “about” means a range of plus or minus 20%, plus or minus 10%, plus or minus 5%, or plus or minus 1% of a given value. Alternatively, particularly with respect to biological systems or processes, the term means within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges

As used herein, the term “nucleic acid construct” can mean a linear polymer of nucleic acids. Said polymers can include promoter sequences that drive the expression of one or more genes of interest. As used herein, the term “nucleic acid construct” can include, but is not limited to, oligonucleotides, RNA, linear DNA, closed end linear DNA, ministrings, transposons, doggybone DNA, GenWand DNA, and minimalistic, immunologically defined gene expression (MIDGE) DNA. As used herein, a nucleic acid construct can be DNA or RNA or a mixture of the two and can comprise naturally occurring or artificial (non-natural) nucleotides. As used herein, “naturally occurring nucleotides” can include adenine, guanine, cytosine, thymine, uracil, inosine, 2,6-diaminopurine, 5-hydroxymethylcytosine, N4-methylated cytosine, N6-methylated adenine, archaeosine and other nucleotide modifications that occur in normal cellular (eukaryotic, prokaryotic or archaea, including virally-induced or phage-induced) metabolism. As used herein, “artificial nucleotide” or “non-natural nucleotide” is a nucleotide analog or linkage that is not naturally occurring and can include, but is not limited to, peptide nucleic acids, morpholinos, phosphorothioate linkages, locked nucleic acids, glycol nucleic acids, addition of functional groups such as amino (—NH2), fluoro (—F), and O-methyl (—OCH3) in the 2′-position of ribose sugar, threose nucleic acids, hexitol nucleic acids, 2′-position sugar modifications, 5-position pyrimidine modifications, 8-position purine modifications, modifications at exocyclic amines, substitution of 4-thiouridine, 5-((3-indolyl)propionamide-N-allyl)-20-deoxyuridine, substitution of 5-bromo or 5-iodo-uracil as well as backbone modifications, anti-reverse cap analogs, substitution of pseudouridine, 5-methylcytidine and/or N1-methyluridine, methylations and unusual base-pairing combinations including, but not limited to, the isobases isocytidine and isoguanidine and (7-(2-thienyl)imidazo[4,5-b]pyridine, Ds).

As used herein, the term “plasmid” can mean a circular DNA molecule that is physically separated from chromosomal DNA. As used herein, the term “plasmid” includes, but is not limited to, bacterially-derived plasmids, minicircles, episomal DNA, covalently closed circular DNA (cccDNA), extrachromosomal circular DNA (eccDNA), chromids, chloroplast DNA, baculovirus-derived circular DNA, bacmids, nanoplasmids, and mitochondrial DNA. The term “plasmid” includes, but is not limited to, small circular, double-stranded DNA molecules with promoter sequences that drive the transcription of one or more genes of interest. Optionally, plasmids can also include an origin of replication (ori) site, marker genes (e.g., antibiotic resistance genes) for selection and/or screening, enhancer elements and restriction endonuclease (RE) sites to allow inserts to be cloned in specific site. As used herein, a plasmid can comprise naturally occurring and/or artificial nucleotides.

As used herein, “antibody” can mean a protein that acts like or is an immunoglobulin or is designed to substitute for an immunoglobulin, including, but not limited to, a polyclonal antibody, a monoclonal antibody, a single-chain variable fragment (scFv), an Fab fragment, a camelid antibody, a nanobody, a Designed Ankyrin Repeat Protein, a monobody, an anticalin, a knottin, an affimer, an avimer, an affinity clamp or an affibody that specifically binds to a substance.

As used herein, “binds selectively”, “bound selectively”, “specifically binds”, “specifically bound”, “specifically recognizes” or “specifically recognized” can mean a substance that binds another substance to the exclusion of others, usually with a dissociation constant (Kd) less than or equal to one (1) micromolar, optionally in an aqueous solution, optionally under specified or defined or strident conditions, for example a temperature ranging from about 23 to about 37 degrees Celsius in a solution having a pH ranging from about 5.5 to about 8, for example, 5.6, 5.7, 5.8, 5.9 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, optionally at a defined salt (for example, sodium chloride) concentration ranging from about 0.0001 to about 10 molar, optionally in a buffer such as a phosphate buffer.

As used herein, the term “subject” or “patient” can refer to any organism to which a composition or formulation in accordance with the disclosure may be administered, e.g., for experimental, diagnostic, prophylactic, and/or therapeutic purposes. Typical subjects include animals (e.g., mice, rats, rabbits, non-human primates, and humans). In some embodiments, a subject is a human. Humans can be more than about: 1, 2, 5, 10, 20, 30, 40, 50, 60, 65, 70, 75, 80, 85, 90, 95, 100, 105, 110, 115 or about 120 years of age. A human may be a pediatric, child, or adult subject.

A “therapeutically effective amount” can mean to an amount of a composition or pharmaceutical composition as disclosed herein with or without additional agents that is effective to achieve its intended purpose, for example to treat a disease. Individual patient needs may vary. Generally, the dosage required to provide an effective amount of the composition will vary, depending on the age, health, physical condition, sex, weight, extent of the disease of the recipient, frequency of treatment and the nature and scope of the disease or condition. For example, a therapeutically effective amount of a composition of pharmaceutical composition herein can range from about 0.0001 mg/kg to about 10000 mg/kg, for example 0.001, 0.01 0.1, 1.0, 10.0 or 100.0, mg/kg, where mg is mg of composition or pharmaceutical composition and kg is kg of bodyweight of a subject or patient.

As used herein, the terms “treatment” or “treating” can mean a pharmaceutical or other intervention regimen for obtaining beneficial or desired results in the recipient. Beneficial or desired results include but are not limited to a therapeutic benefit and/or a prophylactic benefit. A therapeutic benefit refers to eradication or amelioration of one or more symptoms of an underlying disorder being treated. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For a prophylactic benefit, a subject at risk of developing a particular disease, or a subject reporting one or more of the physiological symptoms of a disease may undergo a treatment disclosed herein, even though a diagnosis of this disease may not have been made.

As used herein, “transfection” can refer to the introduction of a nucleic acid construct or plasmid or engineered DNA (e.g., a DNA) into a cell. Transfection can occur, for example, in vitro, ex vivo, or in vivo.

As used herein, “adjuvant” can refer to any substance that promotes a stronger immune response in a subject receiving a vaccine. Examples of adjuvants include, but are not limited to, monophosphoryl lipid A (MPL), oligodeoxynucleotides (ODNs) containing unmethylated CpG motifs (CpG ODN), AS01, AS02, AS03, AS04, MF59, QS-21, Matrix-M, α-Mannosyl Ceramide, D-(+)-trehalose 6,6′-dibehenate, trehalose 6,6-dibehenate, dimethyldioctadecylammonium, glucopyranosyl lipid, and R848.

As used herein, the term “medication package” can refer to a box, packet, bag or plastic that is used to bundle together a Patient Package Insert (PPI), Medication Guide (MG), or Instructions for Use (IFU).

The section headings used herein are for organizational purposes only and are not to be construed as limiting the subject matter described.

### DETAILED DESCRIPTION

### Overview

Disclosed herein are compositions for the prevention and treatment of orthopoxvirus, and compositions containing an antibody that binds to an epitope (with monoepitopic or polyepitopic specificity) found on the intracellular mature virion or mature virion forms of orthopoxviruses and/or an antibody that binds to an epitope found on the extracellular enveloped virion or enveloped virion forms of orthopoxviruses. Also disclosed herein are compositions, including non-blood derived antibody compositions, such as humanized antibodies and those antibodies that have been modified to reduce immunogenicity and/or extend serum half-life, and to methods for their use (such as treatment or prevention of an orthopoxvirus infection) in imparting passive immunity against orthopoxvirus infections to individuals at risk of orthopoxvirus infections or who exhibit vaccinia infection, including adverse events caused by certain smallpox vaccines such as progressive vaccinia and eczema vaccinatum, monkeypox infection, or smallpox infection.

Described herein is also a combination of compositions or pharmaceutical compositions administered separately (concurrently or consecutively), or in a single composition or pharmaceutical composition, of two or more altered and/or humanized monoclonal antibodies (mAbs) that can target viral proteins on the two main forms of the virus: The Mature Virion (MV) which is a non-enveloped particle and is responsible for transmission and the Enveloped Virion (EV) which is an MV particle that has gained an envelope as it buds from an infected host cell and is responsible for cell-to-cell spread within an infected host. When an MV particle is still within an infected host cell, it can be called an Intracellular Mature Virion or IMV. In some instances, the terms MV and IMV can be used interchangeably. When an EV particle releases from an infected host cell, it can be called an Extracellular Enveloped Virion or EEV. When an EV particle initially buds from an infected host cell, but has not yet released from the cell, it can be called a Cell-Associated Enveloped Virion or CEV.

### Antibodies and Functional Fragments Thereof

Provided herein are antibodies and functional fragments thereof that bind to an epitope of an orthopoxvirus. In some embodiments, the epitope is found on a Mature Virion (MV) form of an orthopoxvirus. In some cases, this includes IMV forms of orthopoxviruses. In some embodiments, the epitope is found on an Enveloped Virion (EV) form of an orthopoxvirus. In some cases, this includes EEV and CEV forms of orthopoxviruses. In some embodiments, the antibodies and functional fragments thereof are engineered. In some embodiments, an initial antibody or functional fragment thereof (e.g., mammalian, murine, rat, or rabbit) is selected or a composite engineered antibody is designed. Composite engineered antibodies are built using one, two, three, four, five, or multiple sequence segments derived from variable regions of unrelated antibodies as building blocks. In some embodiments, the initial antibody or the composite engineered antibody is a chimeric antibody. In some embodiments, the initial antibody (e.g., 7d11) is an antibody to a poxvirus (e.g., vaccinia virus) L1 protein. In some embodiments, the initial antibody (e.g., 8A) is an antibody to a poxvirus (e.g., vaccinia virus) ectodomain of the B5 protein. In some embodiments, the initial antibody (e.g., 6C) is an antibody to a poxvirus (e.g., vaccinia virus) ectodomain of the A33 protein. In some embodiments, the engineered antibody or functional fragment thereof comprise a constant region (Fc region) with one or both HC2 and HC3 constant domains.

In some embodiments, the antibodies or functional fragments thereof are altered, which can impart one or more desirable characteristics such as increased half-life of the antibody or functional fragment thereof in a subject. In some embodiments, the antibodies or functional fragments thereof are humanized, which can impart one or more desirable characteristics such as decreased immunogenicity to the antibody or functional fragment thereof in a human subject. In some embodiments, the one or more sequences of the engineered composite antibody or functional fragment thereof is separately or individually humanized. In some instances, the non-human framework sequences can be in an Fc region of an antibody. In some embodiments, the antibody or functional fragment thereof includes a light chain and/or a heavy chain framework region (constant region), such as one or more framework regions (constant regions), including one or more IgG1, IgG2, IgG3 and/or IgG4 framework regions (constant regions).

Non-limiting examples of antibody fragments include orthopoxvirus-binding regions and/or effector regions of the antibody, (e.g., an Fab, an Fab′, an F(ab′)₂, an Fv, an scFv, an (scFv)₂, a dual variable region antibody, a single variable region antibody, a linear antibody, a V region, a multispecific antibody formed from antibody fragments, an F(ab)₂, an Fd, an Fc, a diabody, a di-diabody, a disulfide-linked Fvs (dsFv), a single-domain antibody (e.g., nanobody) or other functional fragments. In general terms, a variable (V) region domain may be any suitable arrangement of heavy (VH) and/or light (VL) chain variable domains). In some instances, each antibody can be, independently, an IgG, an IgA, IgD, IgE, or IgM antibody, or any subclass (e.g., IgG1, IgG2, IgG3, IgG4, IgAQ1, and IgA2), or a biologically active fragment of any of these, for example a Fab fragment, or a light chain or a heavy chain of any of these.

In some embodiments, the antibodies or functional fragments thereof comprise an amino acid sequence that is at least about 80%, at least about 85%, at least about 90%, at least about 95%, at least about 96%, at least about 97%, at least about 98%, at least about 99%, at least about 100% homologous or identical to an amino acid sequence set forth in SEQ ID NOs: 1-27.

### Methods of Expressing and Purifying Antibodies

Antibodies disclosed herein can be made using various methods. The antibodies and functional fragments thereof of the invention can be produced from a host cell. A host cell refers to a vehicle that includes the necessary cellular components, (e.g., organelles), needed to express the polypeptides and constructs described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection). The choice of nucleic acid vectors depends in part on the host cells to be used. Generally, preferred host cells are of either prokaryotic (e.g., bacterial) or eukaryotic (e.g., mammalian) origin.

A nucleic acid sequence encoding the amino acid sequence of an antibody or functional fragment thereof of the invention may be prepared by a variety of methods. These methods include, but are not limited to, oligonucleotide-mediated (or site-directed) mutagenesis and PCR mutagenesis. A nucleic acid molecule encoding the antibody or functional fragment thereof of the invention may be obtained using standard techniques, (e.g., gene synthesis). Alternatively, a nucleic acid molecule encoding a wild-type antibody or functional fragment thereof may be altered to contain specific amino acid substitutions using standard techniques in the art (e.g., mutagenesis). Nucleic acid molecules can be synthesized using a nucleotide synthesizer or PCR techniques.

Nucleic acid sequences encoding an antibody or functional fragment thereof of the invention may be inserted into a vector capable of replicating and expressing the nucleic acid molecules in prokaryotic or eukaryotic host cells. Many vectors are available in the art and can be used for the purpose of the invention. Each vector may contain various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding the protein of interest, and a transcription termination sequence. Vectors can be linearized or supercoiled exhibiting improved transfection efficiency.

In some embodiments, mammalian cells are used as host cells for the invention. In some embodiments, the glutamine-auxotrophic and cholesterol-auxotrophic phenotype is induced by genetic manipulation of a non-glutamine-auxotrophic and non-cholesterol-auxotrophic cell, including for example mutation or deletion of a gene necessary for endogenous glutamine biosynthesis, such as glutamine synthetase. Common methods of genetic engineering are well known to those of skill in the art, e.g., site directed mutagenesis, zinc finger nucleases, shRNA, transposons, See e.g., Cytotechnology. 2007 April; 53(1-3): 65-73. For example, the murine myeloma cells termed NS0 are known glutamine-auxotrophic and cholesterol-auxotrophic cells (See e.g., Barnes et al. Cytotechnology. 2000. Advances in animal cell recombinant protein production: GS-NS0 expression system February; 32(2):109-23; and US 20100028940).

Additional examples of mammalian cell types which may be manipulated to be used as host cells include, but are not limited to, human embryonic kidney (HEK) (e.g., HEK293, HEK 293F), Chinese hamster ovary (CHO), HeLa, COS, PC3, Vero, MC3T3, NS0, VERY, BHK, MDCK, W138, BT483, Hs578T, HTB2, BT20, T47D), CRL7030, and HsS78Bst cells. In other embodiments, E. coli cells are used as host cells for the invention. Examples of E. coli strains include, but are not limited to, E. coli 294 (ATCC 31,446), E. coli λ 1776 (ATCC® 31,537, E. coli BL21 (DE3) (ATCC® BAA-1025), and E. coli RV308 (ATCC© 31,608). Different host cells have characteristic and specific mechanisms for the posttranslational processing and modification of protein products. Appropriate cell lines or host systems may be chosen to ensure the correct modification and processing of the antibody or functional fragment thereof expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of antibodies or functional fragments thereof are known in the art, see, for example, Paulina Balbas, Argelia Lorence (eds.) Recombinant Gene Expression: Reviews and Protocols (Methods in Molecular Biology), Humana Press; 2nd ed. 2004 (Jul. 20, 2004) and Vladimir Voynov and Justin A. Caravella (eds.) Therapeutic Proteins: Methods and Protocols (Methods in Molecular Biology) Humana Press; 2nd ed. 2012 (Jun. 28, 2012).

Host cells used to produce antibodies or functional fragments thereof of the invention may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10% (preferably 8%). The pH of the medium is generally from about 6.8 to 7.4, e.g., 7.0, depending mainly on the host organism. If an inducible promoter is used in the expression vector of the invention, protein expression is induced under conditions suitable for the activation of the promoter. Conventional cell culture conditions for production of antibodies or functional fragments thereof are known in the art, e.g., see Butler, Cell Culture and Upstream Processing, Taylor & Francis; 1st edition (May 25, 2007).

Protein recovery typically involves disrupting the host cell, generally by such means as osmotic shock, sonication, or lysis. Once the cells are disrupted, cell debris may be removed by centrifugation or filtration. The proteins may be further purified. An antibody of the invention may be purified by any method known in the art of protein purification, for example, by protein A affinity, other chromatography (e.g., ion exchange, affinity, and size-exclusion column chromatography), centrifugation, differential solubility, or by any other standard technique for the purification of proteins. (see Process Scale Purification of Antibodies, Uwe Gottschalk (ed.) John Wiley & Sons, Inc., 2009). In some instances, antibodies or functional fragments thereof can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from the influenza hemagglutinin protein.

In some embodiments, the antibodies are designed empirically (i.e., improvements are made through trial and error). In some embodiments, the antibodies are designed in silico using one or more algorithms to optimize one or more features. For example, a database can be created having antibody segments previously screened using immunogenetic assays (e.g., ex vivo T cell immunogenetic assays), having half-life information for each antibody segment, or having MHC Class II binding information. One or more algorithms can then be used to assess the database and predict amino acid alterations that improve one or more desired characteristics such as increasing the half-life of the antibody in a subject, decreasing an immunogenetic response in a subject (e.g., by avoiding sequences that are homologous to T cell epitopes), or both. In some embodiments, the designed antibodies are then codon optimized to minimize the use of rare codons in the coding sequence and improve protein production yields. In some embodiments, the antibodies are codon optimized for expression in mammalian cells (e.g., murine cells).

### Methods of Treatment

Disclosed herein are methods of treatment using a composition or pharmaceutical composition disclosed herein. In some embodiments, the methods described herein can be used in the development of antibodies or functional fragments thereof that can be used to produce medicaments. In some cases, cells can be used in manufacturing of biological products including vaccines and antibodies or functional fragments thereof.

In some embodiments, the methods described herein can be used for antibody or functional fragments thereof delivery in vivo. In some cases, a method can be used herein to deliver an antibody or functional fragment thereof to a subject in need thereof, for example a subject in need of treatment or prevention of a disease (e.g., a viral disease, an orthopoxvirus infection). For example, orthopoxviruses including abatino macacapox virus, akhmeta virus, alaskapox virus, camelpox virus, cowpox virus, ectromelia virus monkeypox virus, raccoonpox virus, skunkpox virus, taterapox virus, vaccinia virus, variola virus, and volepox virus can be treated. In some embodiments, a vaccine comprising a composition or pharmaceutical composition disclosed herein can be administered to a subject to increase immunogenicity in the subject. In some embodiments, a composition or pharmaceutical composition can be used to prevent, ameliorate, and/or reduce the severity of an orthopoxvirus infection.

In some embodiments, the methods, systems, and compositions can be used in an animal models. For example, mice or any mammal can be produced by a method according to the disclosure specifically designed for the study of orthopoxviruses.

In some embodiments, a method of treatment can comprise a therapeutically effective amount of a dose (e.g., a unit dose) of an antibody or functional fragment thereof. In some cases, a composition disclosed herein can be administered to a subject in need of treatment in order to effect treatment. In some cases, a treatment may be preventive and/or therapeutic, and it may be directed to any viral disease (e.g., orthopoxvirus). In some cases, a treatment scheme can be determined by a physician in each case depending on such factors as the orthopoxvirus to be treated, age, and weight of the patient. In some embodiments, the antibody or functional fragments thereof are administered to the patient through a route selected from the group consisting of local, sublingual, buccal, intravenous, subcutaneous, enteral, intra-arterial, intramuscular, intraperitoneal, epidural, intrathecal, intracerebroventricular, intra-articular, intraosseous infusion, intracardiac, intravitreal, parenteral, vaginal, intracavernous, intravesicle, rectal, transdermal, and perivascular.

In some embodiments, a method of treating a patient in need with an antibody or functional fragment thereof can comprise: a first antibody that binds to an epitope found on a Mature Virion (MV) form of viruses of the orthopoxvirus genus; and a second antibody that binds to an epitope found on an Enveloped Virion (EV) form of viruses of the orthopoxvirus genus.

In some embodiments, the present disclosure can comprise a method of treating a patient in need thereof with an antibody or functional fragment thereof comprising: a first antibody that binds to an epitope found on an MV form of viruses of the orthopoxvirus genus; and a second antibody that binds to a first epitope found on an EV form of viruses of the orthopoxvirus genus; and a third antibody that binds to a second epitope found on said EV form of viruses of the orthopoxvirus genus, wherein said first epitope is different than said second epitope.

### Vaccine Applications

In some embodiments, the antibody or functional fragments thereof can be delivered as a vaccine. In some embodiments, the present disclosure can comprise a method of imparting passive immunity against smallpox to a subject, said method comprising administering to said subject an effective amount of a composition comprising: a first antibody that binds to an epitope found on a Mature Virion (MV) form of a variola virus; and a second antibody that binds to an epitope found on an Enveloped Virion (EV) form of a variola virus.

In some embodiments, the present disclosure can comprise a method of imparting passive immunity against infection by a monkeypox virus to a subject, said method comprising administering to said subject an effective amount of a composition comprising: a first antibody that binds to an epitope found on a Mature Virion (MV) form of said monkeypox virus; and a second antibody that binds to an epitope found on an Enveloped Virion (EV) form of said monkeypox virus.

In some embodiments, the present disclosure can comprise a method of imparting passive immunity against smallpox to a subject, said method comprising administering to said subject an effective amount of a composition comprising: a first antibody that binds to an epitope found on a Mature Virion (MV) form of a variola virus; and a second antibody that binds to a first epitope found on an Enveloped Virion (EV) form of said variola virus; and a third antibody that binds to a second epitope found on said EV form of said variola virus, wherein said second epitope is different than said first epitope.

In some embodiments, the present disclosure can comprise a method of imparting passive immunity against infection by a monkeypox virus to a subject, said method comprising administering to said subject an effective amount of a composition comprising: a first antibody that binds to an epitope found on a Mature Virion (MV) form of said monkeypox virus; and a second antibody that binds to a first epitope found on an Enveloped Virion (EV) form of said monkeypox virus; and a third antibody that binds to a second epitope found on said EV form of said monkeypox virus, wherein said second epitope is different than said first epitope.

### Formulations and Excipients, Carriers and Diluents

In some embodiments, compositions disclosed herein can comprise pharmaceutical preparations that include one or more antibodies or functional fragments thereof described herein. In some cases, a composition herein can comprise a pharmaceutical composition. In some cases, a pharmaceutical composition can be in unit dose form. In some cases, a formulation containing the compounds according to the present disclosure can take the form of liquid, solid, semi-solid or lyophilized powder forms, such as, for example, solutions, suspensions, emulsions, or the like, preferably in unit dosage forms suitable for simple administration of precise dosages.

In some embodiments, a pharmaceutical composition can include a conventional pharmaceutical carrier or excipient and can additionally include other medicinal agents, carriers, adjuvants, additives and the like. In some cases, the composition can be about 0.1% to about 85%, about 0.5% to about 75% by weight of an antibody or functional fragment thereof of the disclosure, with the remainder consisting essentially of suitable pharmaceutical excipients. In some embodiments, the amount of active component, e.g., one or more antibodies or functional fragments thereof of the invention included in the pharmaceutical preparations is such that a suitable dose within the designated range is provided (e.g., a dose within the range of 0.01-500 mg/kg of body weight).

Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions of the invention can be administered parenterally in the form of an injectable formulation. Pharmaceutical compositions for injection (i.e., intravenous injection) can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006). In some embodiments, a composition herein can comprise one or more of the following excipients: acacia, acesulfame potassium, acetic acid-glacial, acetone, acetyltributyl citrate, acetyltriethyl citrate, adipic acid, agar, albumin, alcohol, alginic acid, aliphatic polyesters, alitame, allantoin, almond oil, alpha hydroxy acids, alpha tocopherol, aluminum hydroxide adjuvant, aluminum monostearate, aluminum oxide, aluminum phosphate adjuvant, ammonia solution, ammonium alginate, ammonium chloride, argan oil, ascorbic acid, ascorbyl glucoside, ascorbyl palmitate, aspartame, attapulgite, azelaic acid, azulene, bakuchiol, beta glucan, beta-hydroxy-acids, bentonite, benzalkonium chloride, benzethonium chloride, benzoic acid, benzyl alcohol, benzyl benzoate, boric acid, bronopol, butylated glycol, butylated hydroxyanisole, butylated hydroxytoluene, butylene glycol, butylparaben, calcium acetate, calcium alginate, calcium carbonate, calcium chloride, calcium hydroxide, calcium lactate, calcium phosphate-dibasic anhydrous, calcium phosphate-dibasic dihydrate, calcium phosphate-tribasic, calcium silicate, calcium stearate, calcium sulfate, canola oil, capric glycol, capric triglyceride, carbomer, carbon dioxide, carboxymethylcellulose calcium, carboxymethylcellulose sodium, carrageenan, castor oil, castor oil-hydrogenated, cellulose-microcrystalline, cellulose-microcrystalline and carboxymethylcellulose sodium, cellulose-powdered, cellulose-silicified microcrystalline, cellulose acetate, cellulose acetate phthalate, ceramides, ceresin, cetostearyl alcohol, cetrimide, cetearyl alcohol, cetyl alcohol, cetylpyridinium chloride, chitosan, chlorhexidine, chlorobutanol, chlorocresol, chlorodifluoroethane (hcfc), chlorofluorocarbons (cfc), chloroxylenol, cholesterol, citric acid monohydrate, coconut oil, collagen, colloidal silicon dioxide, coloring agents, copper peptide, copovidone, corn oil, corn starch and pregelatinized starch, cottonseed oil, cresol, croscarmellose sodium, crospovidone, cyclodextrins, cyclomethicone, denatonium benzoate, desitin, dextrates, dextrin, dextrose, dibutyl phthalate, dibutyl sebacate, diethanolamine, diethyl phthalate, difluoroethane (hfc), dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol stearates, ethylene vinyl acetate, ethylparaben, fatty acids, ferulic acid, fructose, fumaric acid, gelatin, glucose-liquid, glycerin, glycerol, glyceryl behenate, glyceryl monooleate, glyceryl monostearate, glyceryl palmitostearate, glycine, glycofurol, glycolic acid, glycol stearate, guar gum, hectorite, heptafluoropropane (hfc), hexetidine, hydrocarbons (hc), hyaluronic acid, hydrochloric acid, hydrocortisone, hydrophobic colloidal silica, mesoporous silica, hydroquinone, hydroxyethyl cellulose, hydroxyethylmethyl cellulose, hydroxypropyl betadex, hydroxypropyl cellulose, hydroxypropyl cellulose-low-substituted, hydroxypropyl starch, hypromellose, hypromellose acetate succinate, hypromellose phthalate, imidurea, inulin, iron oxides, isomalt, isoparaffin, isopropyl alcohol, isopropyl myristate, isopropyl palmitate, jojoba oil, kaolin, kojic acid, lactic acid, lactitol, lactose-anhydrous, lactose-inhalation, lactose-monohydrate, lactose-monohydrate and corn starch, lactose-monohydrate and microcrystalline cellulose, lactose-monohydrate and povidone, lactose-monohydrate and powdered cellulose, lactose-spray-dried, lanolin, lanolin-hydrous, lanolin alcohols, lauric acid, lecithin, leucine, linoleic acid, macrogol 15 hydroxystearate, magnesium aluminum silicate, magnesium carbonate, magnesium oxide, magnesium silicate, magnesium stearate, magnesium trisilicate, maleic acid, malic acid, maltitol, maltitol solution, maltodextrin, maltol, maltose, mannitol, medium-chain triglycerides, meglumine, menthol, methionine, methylcellulose, methylparaben, mineral oil, mineral oil-light, mineral oil and lanolin alcohols, monoethanolamine, monosodium glutamate, monothioglycerol, myristic acid, myristyl alcohol, neohesperidin dihydrochalcone, neotame, niacinamide, nitrogen, nitrous oxide, octyldodecanol, oleic acid, oleyl alcohol, olive oil, palmitic acid, paraffin, peanut oil, pectin, PEG-8 stearate, pentetic acid, petrolatum, petrolatum and lanolin alcohols, phenol, phenoxyethanol, phenylethyl alcohol, phenylmercuric acetate, phenylmercuric borate, phenylmercuric nitrate, phospholipids, phosphoric acid, phytic acid, phytosphingosine, polacrilin potassium, poloxamer, polycarbophil, polydextrose, poly (dl-lactic acid), polyethylene glycol, polyethylene oxide, polymethacrylates, poly(methyl vinylether/maleic anhydride), polyoxyethylene alkyl ethers, polyoxyethylene castor oil derivatives, polyoxyethylene sorbitan fatty acid esters, polyoxyethylene stearates, polyoxylglycerides, polyparaben, polysorbate 60, polysorbate 80, polyvinyl acetate phthalate, polyvinyl alcohol, potassium alginate, potassium alum, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, propylparaben sodium, pyrrolidone, raffinose, Retin-A, retinol and retinoic acid derivatives, saccharin, saccharin sodium, safflower oil, salicylic acid, saponite, sesame oil, shellac, simethicone, sodium acetate, sodium alginate, sodium ascorbate, sodium benzoate, sodium bicarbonate, sodium borate, sodium carbonate, sodium chloride, sodium citrate dihydrate, sodium cyclamate, sodium formaldehyde sulfoxylate, sodium hyaluronate, sodium hydroxide, sodium lactate, sodium lauryl sulfate, sodium metabisulfite, sodium phosphate—dibasic, sodium phosphate—monobasic, sodium propionate, sodium starch glycolate, sodium stearyl fumarate, sodium ascorbyl phosphate, sodium deoxycholate, sodium hydroxide, sodium lauroyl lactylate, sodium lauryl sulfate, sodium palmitate, sorbitan stearate, sodium sulfite (E221), sprinolactone, sodium sulfite, sodium thiosulfate, sorbic acid, sorbitan esters (sorbitan fatty acid esters), sorbitan monostearate, sorbitol, soybean oil, sphingomyelins, starch, starch-pregelatinized, starch—sterilizable maize, stearic acid, stearyl alcohol, squalene, sucralose, sucrose, sucrose octaacetate, sugar-compressible, sugar-confectioner's, sugar spheres, sulfobutylether b-cyclodextrin, sulfur dioxide, sulfuric acid, sunflower oil, suppository bases—hard fat, tagatose, talc, tartaric acid, tetrafluoroethane (hfc), thaumatin, thimerosal, thymol, titanium dioxide, tragacanth, trehalose, tretinoin, triacetin, tributyl citrate, tricaprylin, triethanolamine, triethyl citrate, triethanolamine, triolein, undecylenic acid, vanillin, vegetable oil—hydrogenated, vitamins, vitamin e polyethylene glycol succinate, water, wax-anionic emulsifying, wax-carnauba, wax-cetyl esters, wax-microcrystalline, wax-nonionic emulsifying, wax-white, wax-yellow, xanthan gum, xylitol, zein, zinc acetate, and/or zinc stearate.

In some cases, a composition such as a pharmaceutical composition can comprise a carrier or a diluent. In some instances, a carrier or diluent can comprise a water, an alcohol, a salt solution (e.g., saline), or a mixture thereof. In some instances, a carrier can comprise a carbohydrate, a buffer, a salt, a pH adjuster, or any combination thereof. In some cases, a composition herein can comprise a buffering agent, a polymer, an antioxidant, a preservative, a chelating agent, a viscomodulator, a tonicifier, a colorant, an odorant, an opacifier, a suspending agent, a binder, a filler, a plasticizer, a lubricant, or any combination thereof.

In some embodiments, an injectable composition for parenteral administration (e.g. intravenous, intramuscular, or intrathecal) can contain the compound in a suitable intravenous solution, such as sterile physiological salt solution. The composition can also be formulated as a suspension in an aqueous emulsion.

### Administration

In some cases, a pharmaceutical composition can be administered by a method selected from the group consisting of local, oral, sublingual, buccal, mucosal, nasal, intravenous, subcutaneous, enteral, intra-arterial, intramuscular, intraperitoneal, epidural, intrathecal, intracerebroventricular, intra-articular, intraosseous infusion, intracardiac, intravitreal, parenteral, vaginal, intracavernous, intravesicle, rectal, topical, transdermal, inhalation, perivascular, ocular, ear canal, and any combination thereof.

In some cases, administration can comprise delivery of a composition. In some cases, delivery can comprise injection, intravenous administration, subcutaneous administration, intramuscular administration, or a combination thereof. A composition provided herein can be administered by any method. In some instances, a subject can administer the composition in the absence of supervision. In some instances, a subject can administer the composition under the supervision of a medical professional (e.g., a physician, nurse, physician's assistant, orderly, hospice worker). In some cases, a medical professional can administer the composition. In some cases, the subject can administer the composition.

In some embodiments, administering a composition herein can be performed at least about: 1 time per day, 2 times per day, 3 times per day, or more than 4 times per day. In some cases, administering can be performed daily, weekly, monthly, or as needed. In some cases, administration or application of a composition herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In some cases, a treatment duration can be from about: 1 to about 30 days, 1 to about 60 days, 1 to about 90 days, 30 days to about 90 days, 60 days to about 90 days, 30 days to about 180 days, from 90 days to about 180 days, or from 180 days to about 360 days. In some embodiments, administration of a composition disclosed herein can be performed for a treatment duration of at least about 1 week, at least about 2 weeks, at least about 3 weeks, at least about 4 weeks, at least about 1 month, at least about 3 months, at least about 6 moths, at least about 12 months, at least about 1 year or for life. Administration can be performed repeatedly over a lifetime of a subject, such as once a month or once a year for the lifetime of a subject.

In some embodiments, the composition to be administered can contain a quantity of the selected compound in a pharmaceutically effective amount for therapeutic use in a biological system, including a patient or subject according to the present disclosure.

In some embodiments, methods of treating patients or subjects for a particular disease state or infection can comprise administration an effective amount of a pharmaceutical composition comprising an antibody or functional fragment thereof and/or at least one additional bioactive (e.g., antiviral) agent according to the present disclosure. In some cases, the therapeutically effective amount of a composition herein ranges from about 0.000001 mg/kg to about 1000 mg/kg, wherein mg can be mg of the composition and kg can be kg of bodyweight of the subject. For example, a composition of about: 0.000001 mg/kg, 0.00001 mg/kg, 0.0001 mg/kg, 0.001 mg/kg, 0.01 mg/kg, 0.1 mg/kg. 1.0 mg/kg, 10 mg/kg, 100 mg/kg or 1000 mg/kg can be administered to a subject in need thereof.

### Kits

In some embodiments, disclosed herein are kits and containers thereof suitable for performing any one of the methods disclosed herein.

In some embodiments, such a kit can contain a composition comprising an antibody or functional fragment thereof, the composition can comprise: a first antibody that binds to an epitope found on a Mature Virion (MV) form of viruses of the orthopoxvirus genus; and a second antibody that binds to an epitope found on an Enveloped Virion (EV) form of viruses of the orthopoxvirus genus in a container for transport.

In some embodiments, such a kit can contain a composition comprising an antibody or functional fragment thereof comprising: a first antibody that binds to an epitope found on an MV form of viruses of the orthopoxvirus genus; and a second antibody that binds to a first epitope found on an EV form of viruses of the orthopoxvirus genus; and a third antibody that binds to a second epitope found on said EV form of viruses of the orthopoxvirus genus, wherein said first epitope is different than said second epitope in a container for transport.

In some embodiments, such a kit can contain the antibody or functional fragments thereof for delivery as a vaccine. In some embodiments, the kit comprises an effective amount of a composition comprising: a first antibody that binds to an epitope found on a Mature Virion (MV) form of a variola virus; and a second antibody that binds to an epitope found on an Enveloped Virion (EV) form of a variola virus in a container for transport.

In some embodiments, the kit comprises an effective amount of a composition comprising: a first antibody that binds to an epitope found on a Mature Virion (MV) form of a monkeypox virus; and a second antibody that binds to an epitope found on an Enveloped Virion (EV) form of said monkeypox virus in a container for transport.

In some embodiments, the kit comprises an effective amount of a composition comprising: a first antibody that binds to an epitope found on a Mature Virion (MV) form of a variola virus; and a second antibody that binds to a first epitope found on an Enveloped Virion (EV) form of said variola virus; and a third antibody that binds to a second epitope found on said EV form of said variola virus, wherein said second epitope is different than said first epitope in a container for transport.

In some embodiments, the kit comprises an effective amount of a composition comprising: a first antibody that binds to an epitope found on a Mature Virion (MV) form of a monkeypox virus; and a second antibody that binds to a first epitope found on an Enveloped Virion (EV) form of said monkeypox virus; and a third antibody that binds to a second epitope found on said EV form of said monkeypox virus, wherein said second epitope is different than said first epitope in a container for transport.

In some cases, the kit can further comprise a suitable excipient, carrier, diluent, or any combination thereof. In some cases, the excipient, carrier, diluent, or any combination thereof can be any excipient, carrier, diluent, or any combination thereof known for storage of antibodies or functional fragments thereof. Said storage device can be any device known in the art for storing antibodies or functional fragments thereof including, but not limited to, glass vials, plastic vials, pre-filled syringes, and ampules.

### EXAMPLES

### Example 1: Humanization of Murine-Human mAb c7D11

A series of 9 (3 heavy and 3 light chain) humanized heavy and light chain V region sequences were designed entirely from segments of human V region sequences with the objective that T cell epitopes will be avoided. In silico tools for assessing MHC Class II binding, database(s) containing antibody segments previously screened using ex vivo T cell immunogenicity assays were used to design the variants. Following design, three heavy and three light chain variable domain sequences were codon optimized for expression in murine cells, synthesized and cloned into a cloning vector with flanking restriction, for subsequent cloning into expression-appropriate vectors.

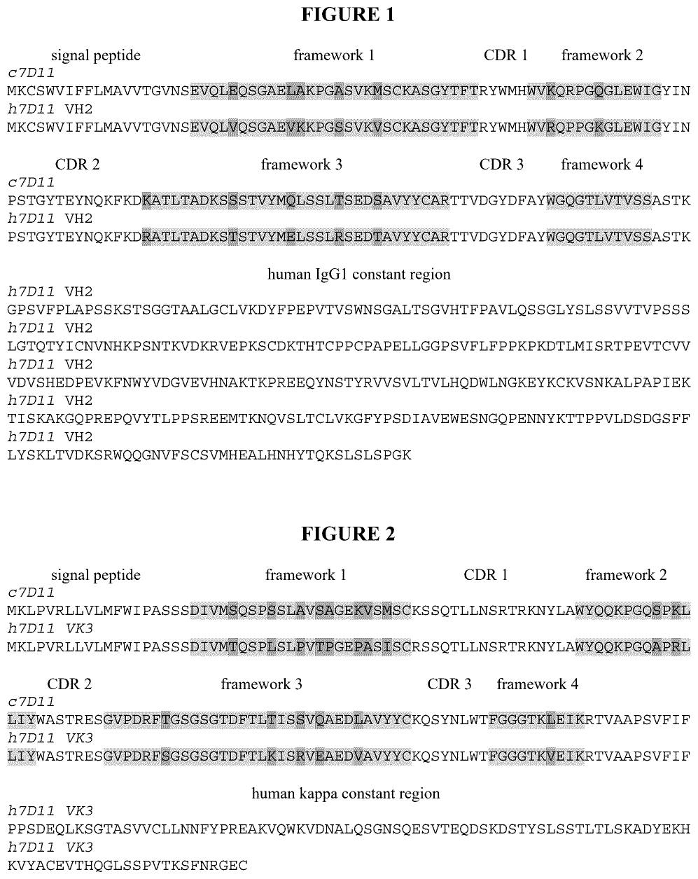

The murine-human c7D11 mAb (SEQ ID NO: 1) was cloned and expressed. FIG. 1 shows a full humanized heavy chain h7D11 variant VH2 amino acid sequence (SEQ ID NO: 3) aligned with the original c7D11 heavy chain chimeric sequence (SEQ ID NO: 2) noting amino acid changes made in the humanized variant VH2. FIG. 2 shows a representative full humanized light chain h7D11 variant VK3 amino acid sequence (SEQ ID NO: 5) aligned with a c7D11 light chain chimeric sequence (SEQ ID NO: 4) noting amino acid changes made in the humanized variant VK3.

### Example 2: Variant c7D11 Expression

Stable NS0 clonal cell lines were generated expressing a properly folded and fully functional IgG consisting of two mature heavy chains with sequence VH2 and two mature light chains with sequence VK3 without the signal peptides for each, which were cleaved during the translocation process in the cells.

### Example 3: Humanization of Chimp-Human Chimeric mAb c8A

A full-size chimp-human mAb was made by fusing the heavy chain variable domain with human IgG1 constant sequences and fusing the light chain variable domain with human lambda constant sequences. This chimeric mAb demonstrated in vitro anti-viral activity against both VACV and VARV in a comet-reduction assay. The mAb also showed protective efficacy in a BALB/c mouse pneumonia model with VACV Western Reserve (WR) lethal challenge.

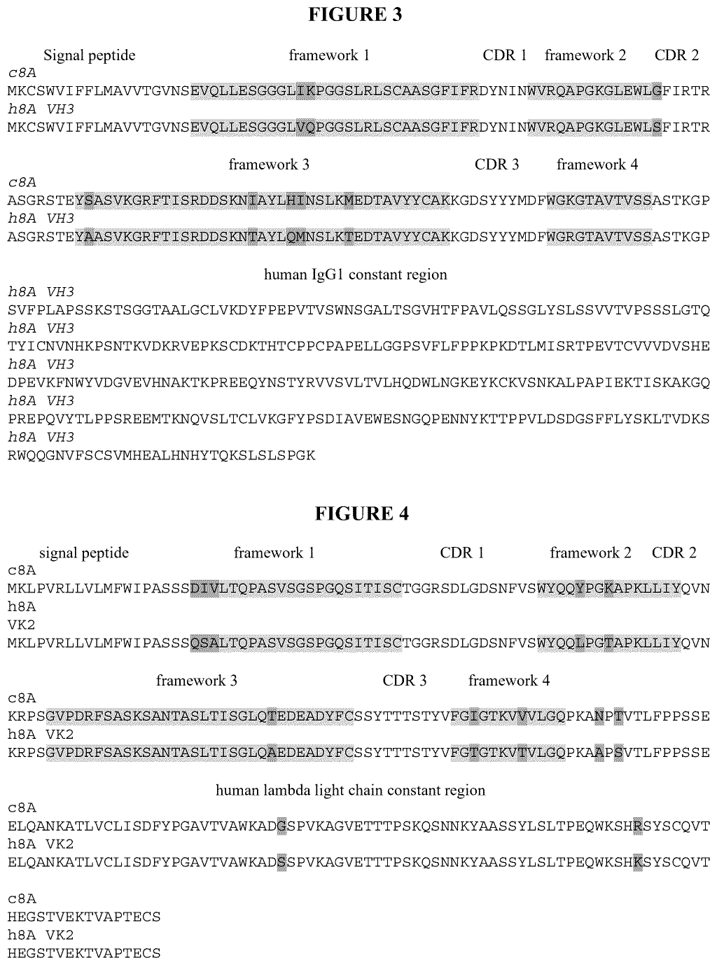

The chimp-human mAb c8A was cloned and expressed (SEQ ID NO: 6). FIG. 3 shows a full humanized heavy chain h8A variant VH3 amino acid sequence (SEQ ID NO: 8) aligned with the original c8A heavy chain chimeric sequence (SEQ ID NO: 7) noting amino acid changes made in the humanized variant VH3. FIG. 4 shows a full humanized light chain h8A variant VK2 amino acid sequence (SEQ ID NO: 10) aligned with the original c8A light chain chimeric sequence (SEQ ID NO: 9) noting amino acid changes made in the humanized variant VK2.

### Example 4: Variant c8A Expression

A stable NS0 clonal cell line, denoted AD2, was generated expressing a properly folded and fully functional IgG having two mature heavy chains with sequence VH3 and two mature light chains with sequence VK2 without the signal peptides, which were cleaved during the translocation process in the cells.

### Example 5: Humanization of Chimp-Human Chimeric mAb c8A

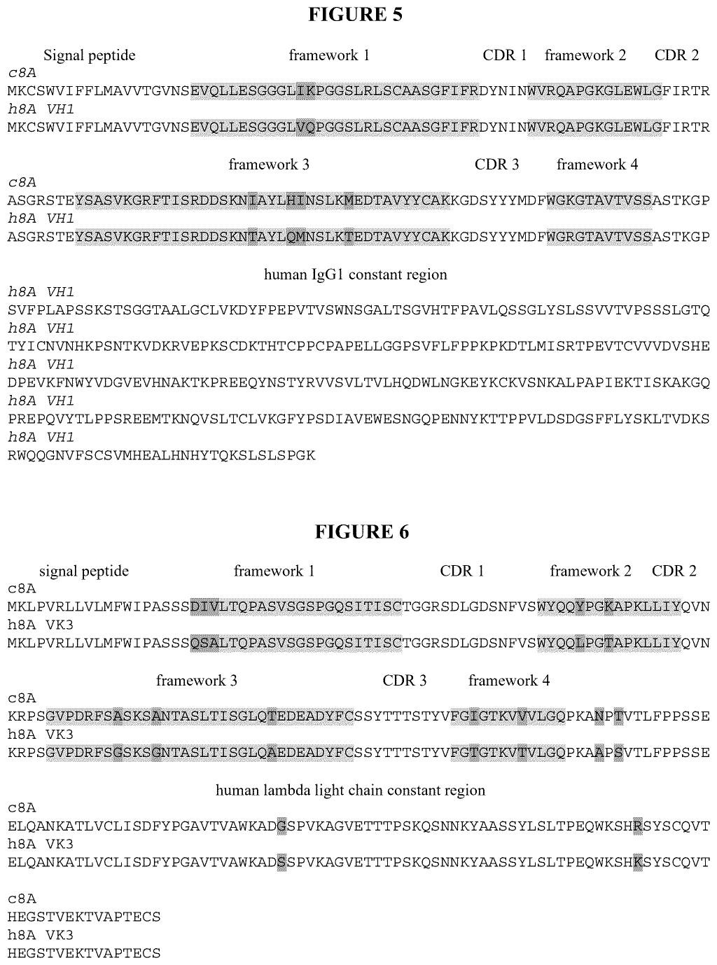

A full-size chimp-human mAb was made by fusing the heavy chain variable domain with human IgG1 constant sequences and fusing the light chain variable domain with human lambda constant sequences. The chimp-human mAb c8A was cloned and expressed (SEQ ID NO: 6). FIG. 5 shows a full humanized heavy chain h8A variant VH1 amino acid sequence (SEQ ID NO: 12) aligned with the original c8A heavy chain chimeric sequence (SEQ ID NO: 11) noting amino acid changes made in the humanized variant VH1. FIG. 6 shows a full humanized light chain h8A variant VK3 amino acid sequence (SEQ ID NO: 14) aligned with the original c8A light chain chimeric sequence (SEQ ID NO: 13) noting amino acid changes made in the humanized variant VK3.

### Example 6: Variant c8A Expression

A stable NS0 clonal cell line, denoted BC9, was generated expressing a properly folded and fully functional IgG consisting of two mature heavy chains with sequence VH1 and two mature light chains with sequence VK3 without the signal peptides, which were cleaved during the translocation process in the cells.

### Example 7: Humanization of Chimp-Human Chimeric mAb c6C

Variable domains were used to create full-size chimp-human mAb by fusing the heavy chain variable domain with human IgG1 constant sequences and fusing the light chain variable domain with human lambda constant sequences. This chimeric mAb demonstrated in vitro anti-viral activity against both VACV and VARV in a comet-reduction assay. The mAb also showed protective efficacy in a BALB/c mouse pneumonia model with VACV Western Reserve (WR) lethal challenge.

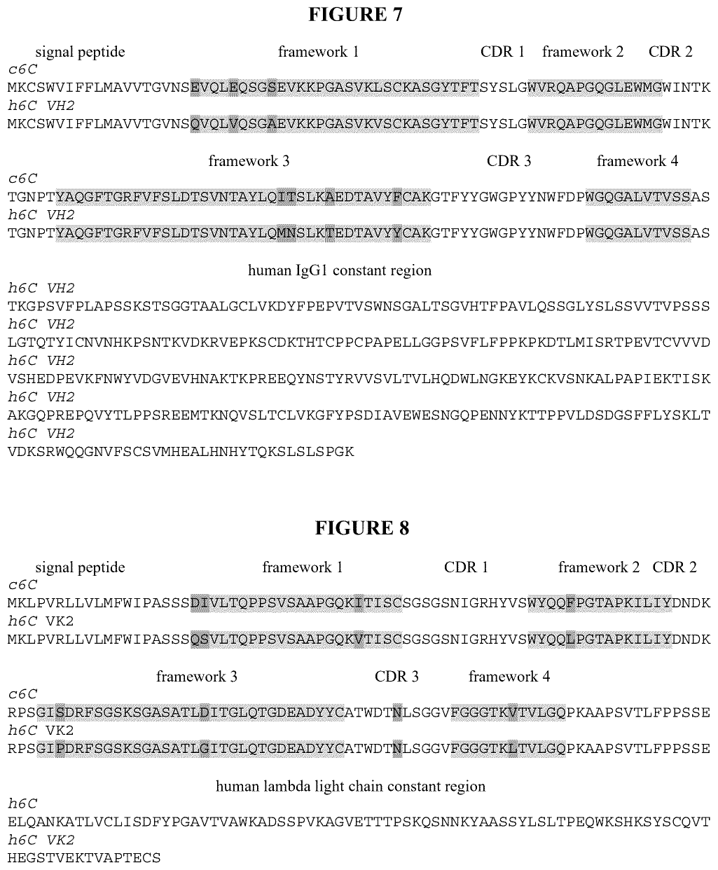

The chimp-human mAb c6C was cloned and expressed (SEQ ID NO: 15). FIG. 7 shows a full humanized heavy chain h6C variant VH2 amino acid sequence (SEQ ID NO: 17) aligned with the original c6C heavy chain chimeric sequence (SEQ ID NO: 16) noting amino acid changes made in the humanized variant VH2. FIG. 8 shows a full humanized light chain h6C variant VK2 amino acid sequence (SEQ ID NO: 19) aligned with the original c6C light chain chimeric sequence (SEQ ID NO: 18) noting amino acid changes made in the humanized variant 2.

### Example 8: Engineering Enhanced Half-Life in Humanized mAbs

The neonatal Fc receptor (FcRn) belongs to the wide range of major histocompatibility complex (MHC) class of molecules whose general function involves antigen presentation. FcRn itself, however, has a narrower scope of function as it is unable to present antigens and instead plays a role in serum half-life via high affinity binding of IgG at low pH. This interaction can be driven by the association of FcRn at the interface of CH2 and CH3 domains in the Fc region of IgG. The engineering of the unmodified humanized 7D11 and 8A mAbs to increase serum half-life can lead to reduction of effective dose and/or number of doses and can lead to more sustained anti-viral potency and protection from orthopoxvirus infections. Consequently, modifications herein can produce Fc variants with increased affinity for FcRn.

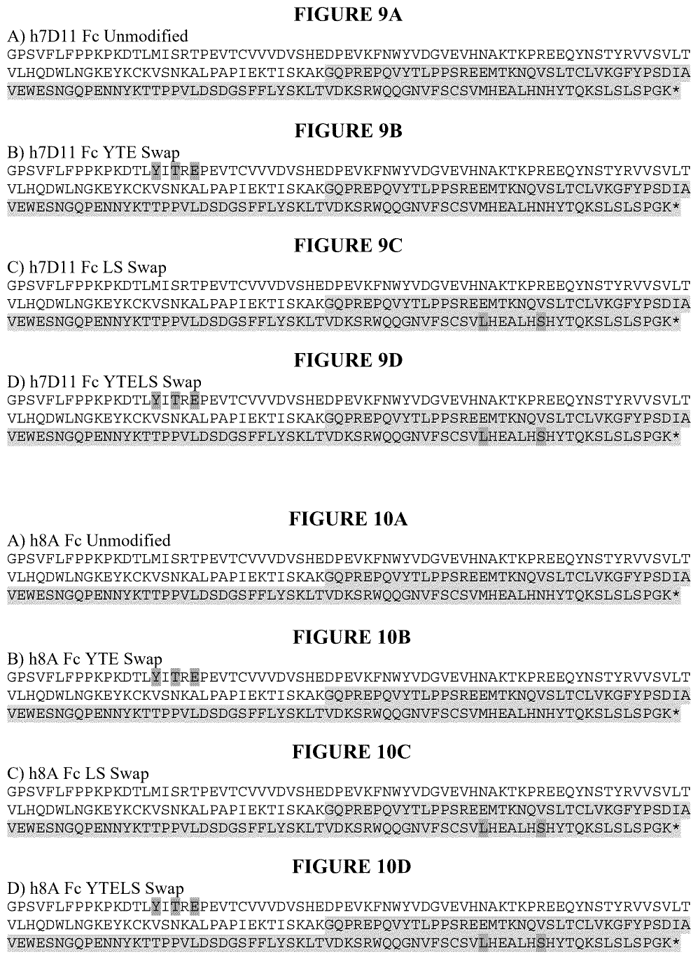

Modifications were made for enhanced affinity of FcRn in h7D11 Heavy Chain Fc Region (SEQ ID NO: 20). FIGS. 9A-9D show variations of the amino acid sequence in the Fc region of h7D11 heavy chain (SEQ ID NOs: 21-23) aligned with an unmodified iteration. Amino acid changes are noted in the variations.

### Example 9: Modifications for Enhanced Affinity of FcRn in h8A Heavy Chain Fc Region

Modifications were made for enhanced affinity of FcRn in h8A Heavy Chain Fc Region (SEQ ID NO: 24). FIGS. 10A-10D show variations of the amino acid sequence in the Fc region of h8A heavy chain (SEQ ID NOs: 25-27) aligned with an unmodified iteration. Amino acid changes are noted in the variations. It is also important to note that while the Fc region is consistent between h7D11 and h8A, the amino acid changes, while identical, are located at different positions due to a 2 amino acid difference in the variable regions of the respective heavy chains.

### Example 10: Drug Product Composition

A combination of at least one anti-MV and one anti-EV can form an effective and efficacious drug product. Disclosed is a combination, which can be in a pharmaceutical composition, of mAbs h7D11 and h8A, mAbs h7D11 and h6C, or a triple combination of mAb h7D11, mAb h8A, and mAb h6C. The variants of each of the mAb components can be selected from those disclosed herein to include variants of mAb h7D11 and mAb h8A with modifications imparting enhanced serum half-life as described herein. A pharmaceutical composition can be in the form of a liquid, a frozen composition or form, or a lyophilized composition or form. The amount of any antibody or fragment thereof, can be present, independently, for example in a composition or pharmaceutical composition, in an amount ranging from about 0.0001 mg to about 10,000 mg, for example about: 0.001 mg, 0.01 mg, 0.1 mg, 1 mg, 5 mg, 10 mg, 20 mg, 30 mg, 40 mg, 50 mg, 100 mg, 500 mg, 1,000 mg, 5,000 mg, or 10,000 mg. An antibody or functional fragment thereof can be administered to a subject in an amount ranging from about 0.001 mg per kg to about 200 mg per kg, where mg per kg is mg of antibody or functional fragment thereof and kg is kg of bodyweight of the subject. Antibodies can be administered, for example, intravenously, subcutaneously, intramuscularly, as separate formulations that are administered consecutively or concurrently, or administered as part of a pharmaceutical composition comprising a pharmaceutically acceptable: excipient, diluent, carrier, or any combination of these. Administration can be 1, 2, 3, 4, or 5 times per day, for example, and administration can be about: once a day, once a week, once every two weeks, once every three weeks, once a month, once every two months, once every three months, or as needed. In some embodiments, when two or more antibodies, which can be monoclonal antibodies (mAbs), are to be administered to a subject, each antibody or mAb can be mixed at the time of administration or given as separate injections either intramuscularly, subcutaneously or intravenously; concurrently or consecutively. Any pharmaceutical or antibody composition or formulation can be in unit dose form. A subject can be a mammal, which can be a human. A subject can be a subject in need thereof. Compositions, pharmaceutical compositions, antibodies, and functional fragments thereof can be contained in a container such as a bag, for example, an intravenous bag, or a syringe, thereby forming a kit.

### Example 11: High-Concentration Drug Product Formulation for Intramuscular Administration

To deliver, for example, an estimated dose of 10-200 mg of total IgG per kg of bodyweight of a subject with an intra-muscular dose volume of 5 ml per injection, a concentration of >100 mg of total IgG/ml may be effective. In some embodiments, the subject is a mammal. In some embodiments, the subject is a human. Histidine, sucrose, and polysorbate-80 can be used individually or in any combination as components for the monoclonal antibody formulations. An average pH for the monoclonal antibody formulations can be 6.0±0.4. A stable formulation at pH 6.0 can have histidine (buffer), sucrose (stabilizer and excipient), and polysorbate-80 (stabilizer) was developed. In addition to a liquid formulation, to minimize injection volume, the composition can be filled with the target dose amount and lyophilized. Prior to administration, the lyophilized drug product can be reconstituted to half the original fill volume. Similar formulations may be used for subcutaneously or intravenously delivered antibodies, functional fragments thereof, compositions including these, or pharmaceutical compositions including these.

### Example 12: Uses of the Compositions

Indications for antibodies and compositions herein can include: i) post-exposure prophylaxis for monkeypox in adults and pediatric individuals who are at high risk for progression to symptomatic monkeypox; ii) treatment of monkeypox in adults and pediatric patients with positive results of monkeypox viral testing, and who are at high risk for progression to symptomatic monkeypox; and iii) pre-exposure prophylaxis, for certain adults and pediatric individuals with no known monkeypox virus exposure, who either have moderate to severely compromised immune systems or for whom vaccination with any available monkeypox vaccine is not recommended due to a history of severe adverse reaction to a monkeypox vaccine(s) and/or monkeypox vaccine component(s).

Medical conditions or treatments that may result in moderate to severe immunocompromise and an inadequate immune response to Monkeypox vaccination include but are not limited to: i) moderate or severe primary immunodeficiency (e.g., DiGeorge syndrome, Wiskott-Aldrich syndrome); ii) active treatment for solid tumor and hematologic malignancies receiving chemotherapeutic agents classified as severely immunosuppressive; iii) receipt of solid-organ transplant and taking transplant-related immunosuppressive drugs; iv) receipt of chimeric antigen receptor (CAR)-T-cell or hematopoietic stem cell transplant (within 2 years of transplantation or taking immunosuppression therapy); v) advanced or untreated HIV infection; or vi) active treatment with high-dose corticosteroids (e.g., >20 mg prednisone or equivalent per day when administered for >2 weeks), alkylating agents, tumor necrosis factor (TNF) blockers, and other biologic agents that are immunosuppressive or immunomodulatory (e.g., B-cell depleting agents).

### Example 13: Dosage and Administration

A dosage for pre-exposure prophylaxis, emergency use in the treatment of monkeypox in adults and pediatric patients, and post-exposure prophylaxis, are determined in non-human animal studies and are verified in Phase 1 human trials in healthy adult volunteers. The dose may be administered as two separate consecutive intramuscular injections into each gluteal muscle, as a single intramuscular injection containing two antibodies into one gluteal muscle, or via intravenous infusion.

An injection can include a pharmaceutical composition comprising an active pharmaceutical ingredient (API) humanized mAb h7D11 with or without engineered half-life modifications in a single-dose vial as a liquid formulation or to be reconstituted from a lyophilized state. The injection can also include a pharmaceutical composition comprising active pharmaceutical ingredient (API) humanized mAb h8A with or without engineered half-life modifications in a single-dose vial as a liquid formulation or to be reconstituted from a lyophilized state. Two or more antibodies or functional fragments thereof may be combined into a single-dose vial as a liquid formulation or to be reconstituted from a lyophilized state.

Antibodies and compositions herein can be contraindicated in individuals with previous severe hypersensitivity reactions, including anaphylaxis, to any component of the antibodies or compositions.

Other indications can include pre-exposure prophylaxis of monkeypox in adults and pediatric individuals (12 years of age and older weighing at least 40 kg): i) who are not currently infected with Monkeypox and who have not had a known recent exposure to an individual infected with Monkeypox; ii) who have moderate to severe immune compromise due to a medical condition or receipt of immunosuppressive medications or treatments and may not mount an adequate immune response to Monkeypox vaccination; or iii) for whom vaccination with any available approved or authorized Monkeypox vaccine, is not recommended due to a history of severe adverse reaction (e.g., severe allergic reaction) to a Monkeypox vaccine(s) and/or Monkeypox vaccine component(s).

Other indications can include post-exposure prophylaxis in adults and pediatric individuals (12 years of age and older weighing at least 40 kg), who are at high risk for progression to severe Monkeypox disease, including hospitalization or death.

Other indications can include treatment of active monkeypox disease in adults and pediatric individuals (12 years of age and older weighing at least 40 kg), with laboratory-confirmed monkeypox infection with positive results of monkeypox viral testing, and who are at high risk for imminent progression to symptomatic monkeypox, and in symptomatic or asymptomatic patients whom other forms of treatment are contraindicated or deemed suboptimal.

A randomized, double-blinded, placebo-controlled trial in adults determines the pre-exposure prophylaxis dose. A primary endpoint confirms a significant reduction in incidence of Monkeypox positive symptomatic illness for participants who receive a single IM or IV dose compared to placebo at a median follow-up time determined on the half-life of a drug product.

Repeat dosing could be needed for a pre-exposure prophylaxis authorization to ensure prophylactic effects for individuals in whom long-term protection is determined to be appropriate (e.g., immunocompromised or comorbidities). Maintenance dosing to maintain similar or slightly greater drug exposures to those observed close to the timepoint where efficacy is determined for the single dose is extrapolated from clinical trial data. Safety may differ with repeat dosing based on hypersensitivity reactions and development of anti-drug antibodies.

### Example 14: Dosage and Administration in Mice

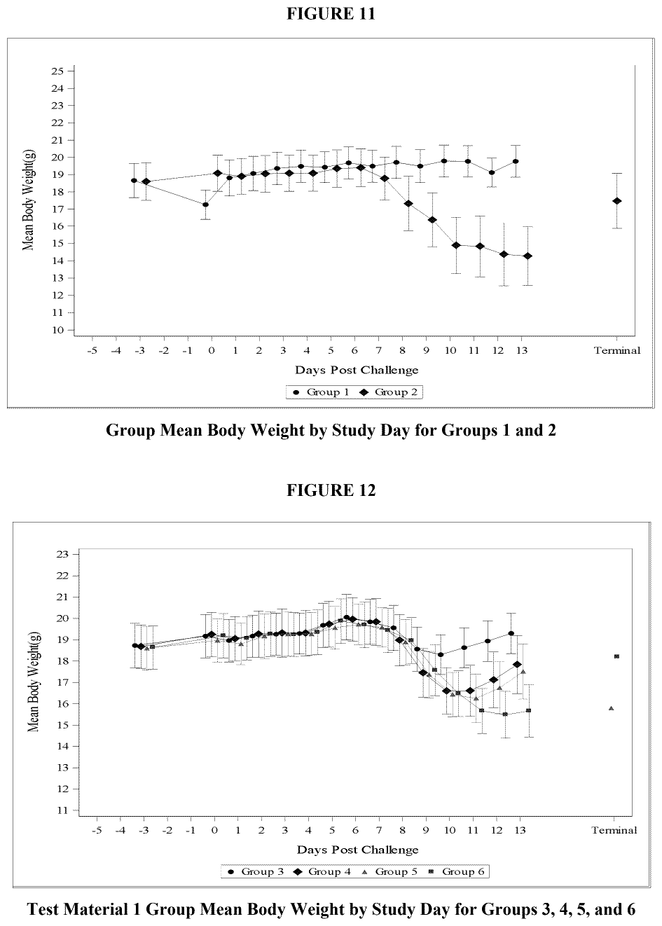

Group 1 and 2 mice (10 male and 10 female BALBc mice; n=20) were used as control groups. Group 1 was no Ectromelia virus (ECTV) challenge with vehicle treatment and group 2 was challenged with 200 PFU of ECTV with vehicle treatment. FIG. 11 shows that group 1 mice did not have a change in mean body weight upon challenge and group 2 had a moderate change in mean body weight, indicating that the health of the animal is affected by viral challenge, but not by vehicle treatment.

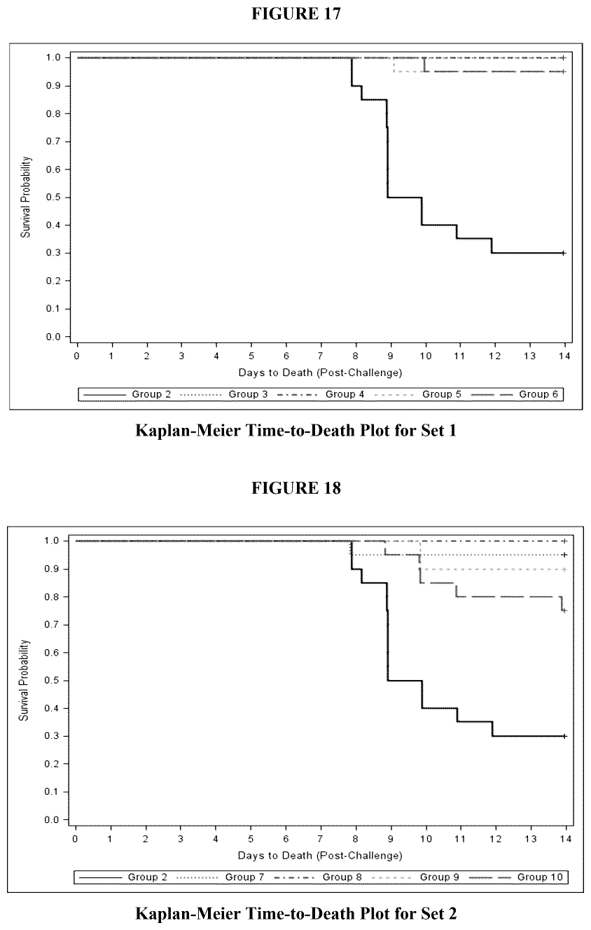

Groups 3, 4, 5, and 6 were challenged and dosed. Mice were challenged with 200 PFU of ECTV on Day 0. Groups 3, 4, 5 and 6 were treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 3 was treated on Day 3 post-challenge, Group 4 was treated on Day 4 post-challenge, Group 5 was treated on Day 5 post-challenge, and Group 6 was treated on Day 6 post-challenge. FIG. 12 shows that group 3 lost the least amount of weight followed by groups 4, 5, and 6. This shows that earlier treatment upon exposure to ECTV had a more pronounced benefit of treatment. FIG. 17 shows that the survival probability was greater than 90% for all treatment time points compared to a 30% survival probability for group 2 control.

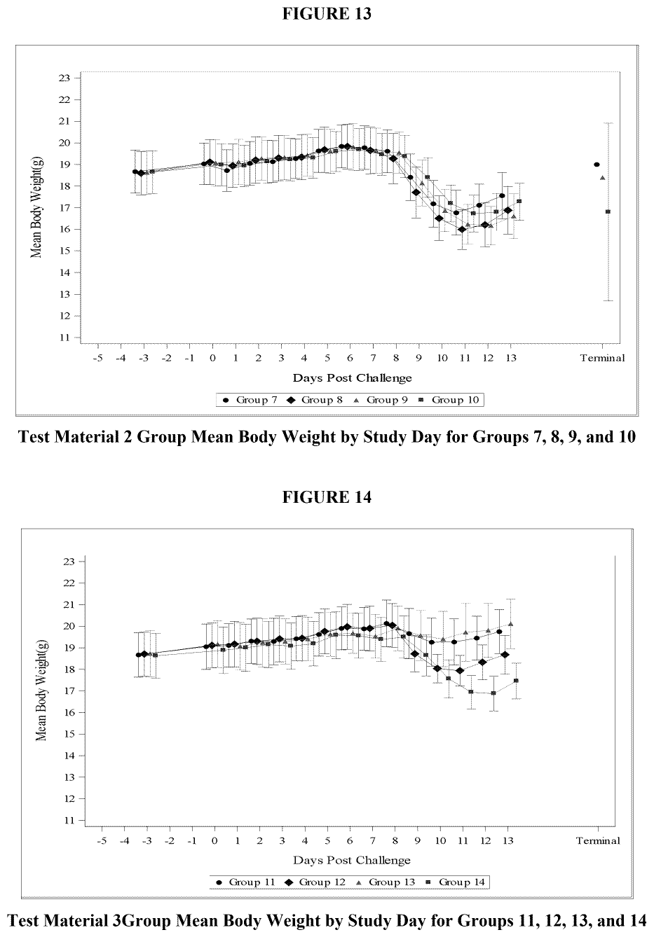

Groups 7, 8, 9, and 10 were challenged and dosed. Mice were challenged with 200 PFU of ECTV on Day 0. Groups 7, 8, 9 and 10 were treated with a cocktail of 5 mg/kg h7D11 (anti-L1 mAb) and 5 mg/kg h8A (anti-B5 mAb) for a 10 mg/kg total mAb cocktail dose. Group 7 was treated on Day 3 post-challenge, Group 8 was treated on Day 4 post-challenge, Group 9 was treated on Day 5 post-challenge, and Group 10 was treated on Day 6 post-challenge. FIG. 13 shows that group 7 lost the least amount of weight followed by groups 10, 8, and 9. This shows generally that earlier treatment upon exposure to ECTV had a more pronounced benefit of treatment. FIG. 18 shows that the survival probability was greater than 90% for treatment time points of groups 7 and 8 followed by 90% for group 9 and 70% for group 10 compared to a 30% survival probability for group 2 control.

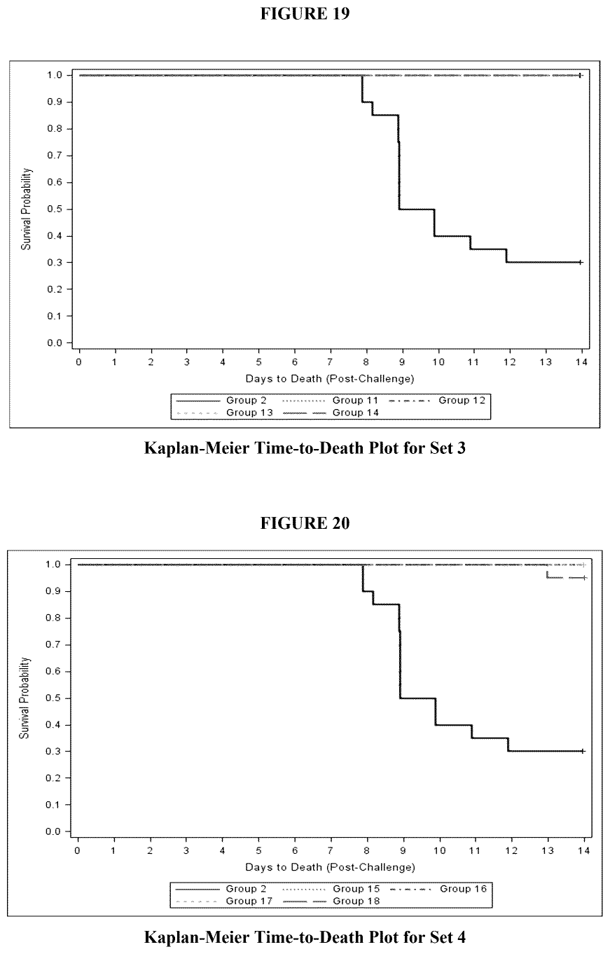

Groups 11, 12, 13, and 14 were challenged and dosed. Mice were challenged with 200 PFU of ECTV on Day 0. Groups 11, 12, 13 and 14 were treated with a cocktail of 5 mg/kg h7D11 (anti-L1 mAb) and 5 mg/kg h6C (anti-A33 mAb) for a 10 mg/kg total mAb cocktail dose. Group 11 was treated on Day 3 post-challenge, Group 12 was treated on Day 4 post-challenge, Group 13 was treated on Day 5 post-challenge, and Group 14 was treated on Day 6 post-challenge. FIG. 14 shows that group 13 lost the least amount of weight followed by groups 11, 12, and 14. This shows generally that earlier treatment upon exposure to ECTV had a more pronounced benefit of treatment. FIG. 19 shows that the survival probability was 100% for all treatment time points compared to a 30% survival probability for group 2 control.

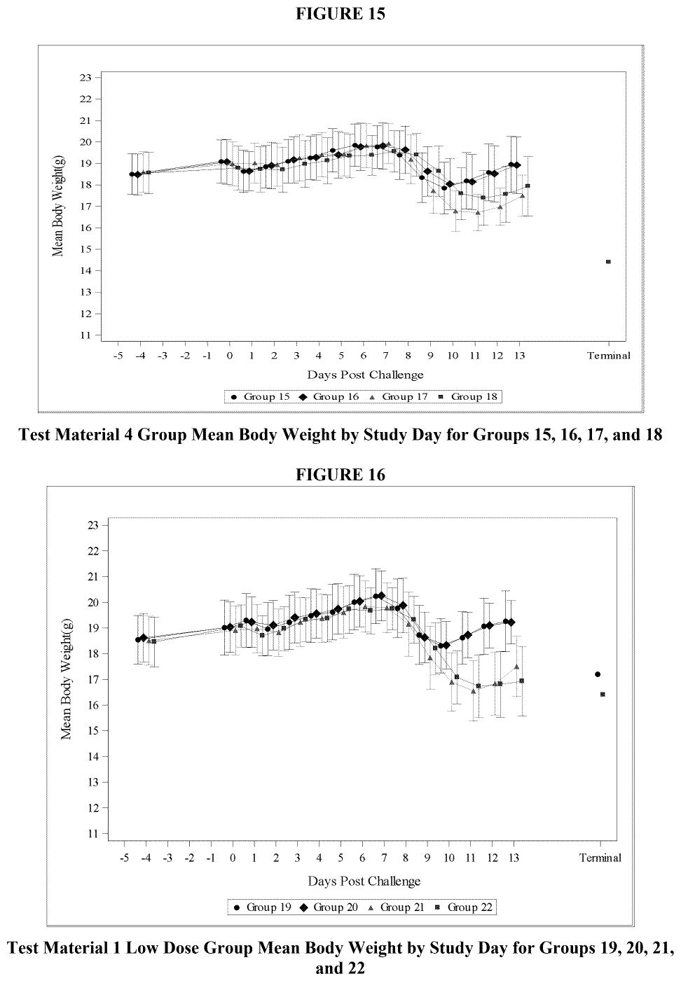

Groups 15, 16, 17, and 18 were challenged and dosed. Mice were challenged with 200 PFU of ECTV on Day 0. Groups 15, 16, 17 and 18 were treated with a cocktail of the original chimeric versions of the mAbs as a comparator to the humanized versions used in all other treatment groups at 5 mg/kg c7D11 (anti-L1 mAb), 5 mg/kg c8A (anti-B5 mAb) and 5 mg/kg c6C (anti-A33 mAb) for a 15 mg/kg total mAb cocktail dose. Group 15 was treated on Day 3 post-challenge, Group 16 was treated on Day 4 post-challenge, Group 17 was treated on Day 5 post-challenge, and Group 18 was treated on Day 6 post-challenge. FIG. 15 shows that group 15 lost the least amount of weight followed by groups 16, 18, and 17. This shows generally that earlier treatment upon exposure to ECTV had a more pronounced benefit of treatment. FIG. 20 shows that the survival probability was greater than 90% for all treatment time points compared to a 30% survival probability for group 2 control.

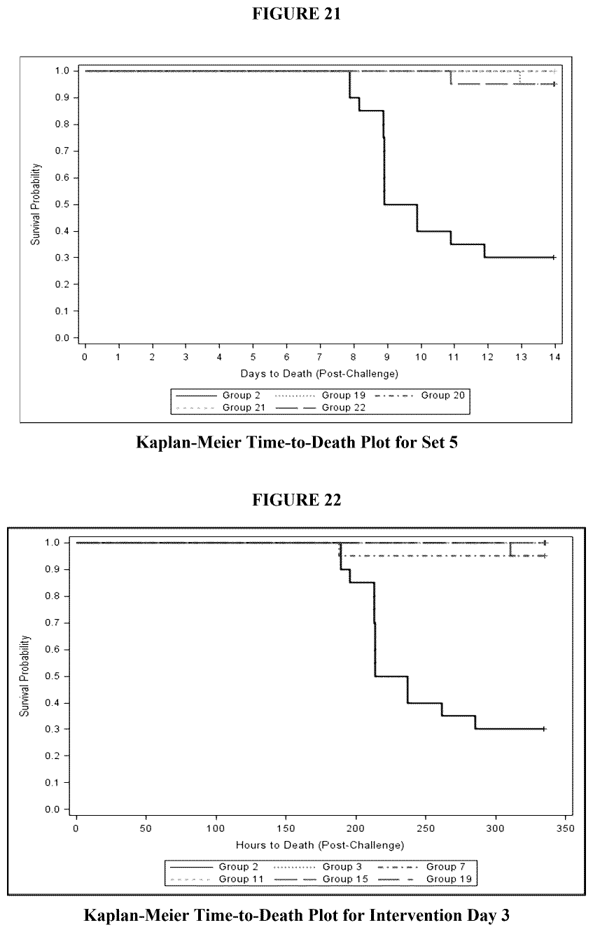

Groups 19, 20, 21, and 22 were challenged and dosed. Mice were challenged with 200 PFU of ECTV on Day 0. Groups 19, 20, 21 and 22 were treated with a cocktail of 3.33 mg/kg h7D11 (anti-L1 mAb), 3.33 mg/kg h8A (anti-B5 mAb) and 3.33 mg/kg h6C (anti-A33 mAb) for a 10 mg/kg total mAb cocktail dose. Group 19 was treated on Day 3 post-challenge, Group 20 was treated on Day 4 post-challenge, Group 21 was treated on Day 5 post-challenge, and Group 22 was treated on Day 6 post-challenge. FIG. 16 shows that groups 19 and 20 lost the least amount of weight followed by groups 21 and 22. This shows generally that earlier treatment upon exposure to ECTV had a more pronounced benefit of treatment. FIG. 21 shows that the survival probability was greater than 90% for all treatment time points compared to a 30% survival probability for group 2 control.

Groups 2, 3, 4, 5, and 6 were challenged and dosed. Mice were challenged with 200 PFU of ECTV on Day 0. Group 2 (negative control) was treated with vehicle only on Day 3 post-challenge. Groups 3, 4, 5 and 6 were treated with a cocktail of 5 mg/kg h7D11 (anti-L1 mAb), 5 mg/kg h8A (anti-B5 mAb) and 5 mg/kg h6C (anti-A33 mAb) for a 15 mg/kg total mAb cocktail dose. Group 3 was treated on Day 3 post-challenge, Group 4 was treated on Day 4 post-challenge, Group 5 was treated on Day 5 post-challenge, and Group 6 was treated on Day 6 post-challenge.

FIG. 22 shows a comparison of day 3 intervention for the compositions compared to group 2 control. The survival probability for groups 3, 7, 11, 15, and 19, which received treatment at day 3, was greater than 90% for all treatment groups compared to a 30% survival probability for group 2 control.

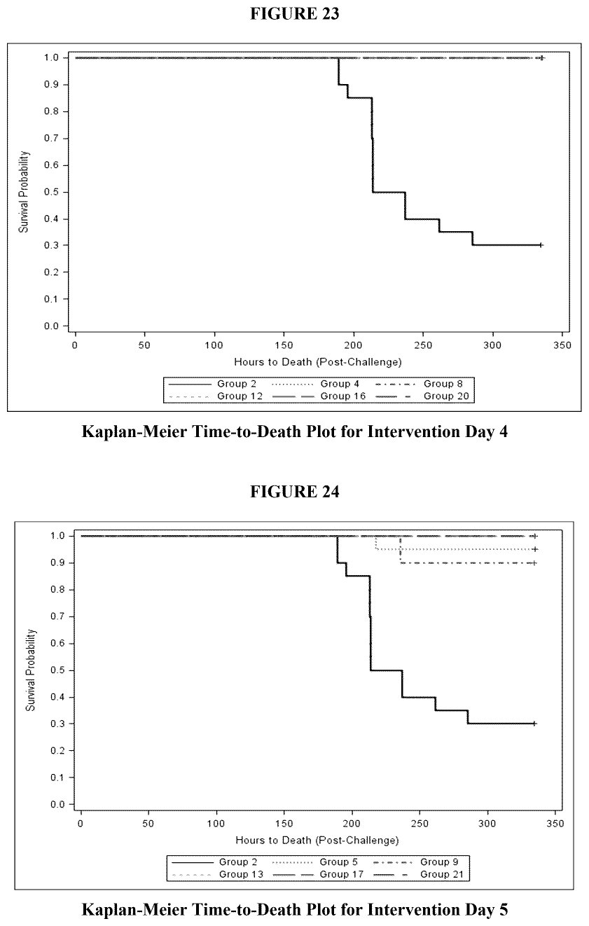

FIG. 23 shows a comparison of day 4 intervention for the compositions compared to group 2 control. The survival probability for groups 4, 8, 12, 16, and 20, which received treatment at day 4, was 100% for all treatment groups compared to a 30% survival probability for group 2 control.

FIG. 24 shows a comparison of day 5 intervention for the compositions compared to group 2 control. The survival probability for groups 5, 9, 13, 17, and 21, which received treatment at day 5, was equal to or greater than 90% for all treatment groups compared to a 30% survival probability for group 2 control.

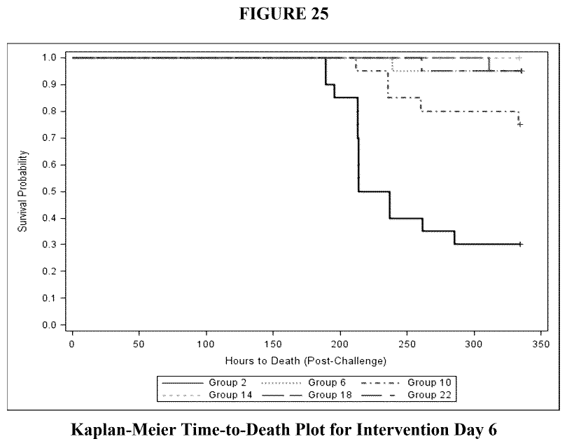

FIG. 25 shows a comparison of day 6 intervention for the compositions compared to group 2 control. The survival probability for groups 6, 10, 14, 18, and 22, which received treatment at day 6, was greater than 90% for all treatment groups except for group 10, which was 70%, compared to a 30% survival probability for group 2 control.

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define a scope of the disclosure and that methods and structures within the scope of these claims and their equivalents be covered thereby.

1. A composition comprising:
(A) a first antibody that binds to an epitope found on a Mature Virion (MV) form of viruses of the orthopoxvirus genus; and
(B) a second antibody that binds to an epitope found on an Enveloped Virion (EV) form of viruses of the orthopoxvirus genus, wherein the epitope found on the EV form of the virus is an epitope of a vaccinia virus B5 protein or an epitope of a vaccinia virus A33 protein.

2. The composition of claim 1, wherein said epitope found on said MV form of said viruses is an epitope of a vaccinia virus L1 protein.

3. The composition of claim 2, wherein said epitope of said L1 protein is located within a domain of said L1 protein that has an amino acid sequence of SEQ ID NO 1.

4. The composition of claim 3, wherein said first antibody is a humanized 7D11 antibody.

5. The composition of claim 3, wherein said first antibody is a humanized 7D11 antibody variant comprising a light chain amino acid sequence of SEQ ID NO: 5 and a heavy chain amino acid sequence of SEQ ID NO: 3.

6. The composition of claim 5, wherein said humanized 7D11 antibody variant comprises an h7D11 Fc YTE Swap modification that comprises an Fc HC2 and HC3 domain with an amino acid sequence of SEQ ID NO 21.

7. The composition of claim 5, wherein said humanized 7D11 antibody variant comprises an h7D11 Fc LS Swap modification that comprises an Fc HC2 and HC3 domain with an amino acid sequence of SEQ ID NO 22.

8. The composition of claim 5, wherein said humanized 7D11 antibody variant comprises an h7D11 Fe YTELS Swap modification comprising that comprises an Fc HC2 and HC3 domain with an amino acid sequence of SEQ ID NO 23.

9. (canceled)

10. The composition of claim 1, wherein said epitope of said B5 protein is located within a domain of said B5 protein that has an amino acid sequence of SEQ ID NO 6.

11. The composition of claim 10, wherein said second antibody is a humanized 8A antibody.

12. The composition of claim 10, wherein said second antibody is a humanized 8A antibody variant comprising a light chain amino acid sequence of SEQ ID NO 10 and a heavy chain amino acid sequence of SEQ ID NO 8.

13. The composition of claim 12, wherein said humanized 8A antibody variant comprises an h8A Fc YTE Swap modification comprising an Fc HC2 and HC3 domain with an amino acid sequence of SEQ ID NO 25.

14. The composition of claim 12, wherein said humanized 8A antibody variant comprises an h8A Fc LS Swap modification comprising an Fc HC2 and HC3 domain with an amino acid sequence of SEQ ID NO 26.

15. The composition of claim 12, wherein said humanized 8A antibody variant comprises an h8A Fc YTELS Swap modification comprising an Fc HC2 and HC3 domain with an amino acid sequence of SEQ ID NO 27.

16. The composition of claim 1, wherein said second antibody is a humanized 8A antibody variant comprising the light chain amino acid sequence of SEQ ID NO 14 and the heavy chain amino acid sequence of SEQ ID NO 12.

17. (canceled)

18. The composition of claim 1, wherein said epitope of said A33 protein is located within a domain of said A33 protein having an amino acid sequence of SEQ ID NO 15.

19. The composition of claim 18, wherein said second antibody is a humanized 6C antibody.

20. The composition of claim 18, wherein said second antibody is a humanized 6C antibody variant comprising a light chain amino acid sequence of SEQ ID NO 19 and a heavy chain amino acid sequence of SEQ ID NO 17.

21.-129. (canceled)
